SEQUENCE LISTING (25 sequences)

SEQ ID NO: 1 - DNA, 407 bp, Homo sapiens
ctctgattttaaaggaagtagatacttcaaataattcatcatggagtgcaatattttctgtaggcttttagtagataacttcatcagtttaaagaagatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgacattgtatgtatttcagtttgcaaatattggactaggatttttgtatctatattccttagtttgacctgtaaattttatttcttgtactaagtattagcctcacgaaaggcattgtcaaatgtt

SEQ ID NO: 2 - DNA, 770 bp, Homo sapiens
gggtctccaacttgccaactgtagatcttggaacctttcatccttcataactgcataagccaattccttctaataaatctgtataatatatctgtctatataataaatatgtacttacataactctgtatgttacatctatctattctatctatctatctatctatctatctatctatctatctatcatctatctatctatttatctattctctgtgtctttggagaaccctgacatagtaagcaatcatatcacctgcaaatgatgaaagctgtgtattttccaaatcagtcgttttatgtctttttttcttgcactgactagtgccccctagagggaatgataattggaattattgtcttgctctgattttaaaggaagtagatacttcaaataattcatcatggagtgcaatattttctgtaggcttttagtagataacttcatcagtttaaagaagatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgacattgtatgtatttcagtttgcaaatattggactaggatttttgtatctatattccttagtttgacctgtaaattttatttcttgtactaagtattagcctcacgaaaggcattgtcaaatgtt

SEQ ID NO: 3 - DNA, 444 bp, Homo sapiens
atctattctctgtgtctttggagaaccctgacatagtaagcaatcatatcacctgcaaatgatgaaagctgtgtattttccaaatcagtcgttttatgtctttttttcttgcactgactagtgccccctagagggaatgataattggaattattgtcttgctctgattttaaaggaagtagatacttcaaataattcatcatggagtgcaatattttctgtaggcttttagtagataacttcatcagtttaaagaagatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgac

SEQ ID NO: 4 - DNA, 429 bp, Mus musculus
aaaacataccactttagtttaaggactatagtgattccacactaggtaaggtgctttctgtaggcttttagttaatagttttgtcaagctaaagaagatctccagatggctaaacttttaaatcatgaatgaagtagatattaccaaattgctttttcagcatccatttagataatcatgttttttgcctttaatctgttaatgtagtgaattacagaaatacatttcctaaatcattacatcccccaaatcgttaatctgctaaagtacatctctggctcaaacaagactggttgtgacaggtttgtctctgtcagtttgtgactgttgggctggctcttcctacccctctgcttcttggtttggcctgaacattaattttattttatttttttaattttacctacaatcaatttcacaatgtgtgtt

SEQ ID NO: 5 - DNA, 792 bp, Mus musculus
ggtctcaacaagatacaaattatgttctctaggtagcaattaacacaaggaacgccttgaggtatgggaggggtgaggaagctcacaagatagaccctggtgcctggaaggaagacagccaactaaaggtcatatcacagtgtcccgggaaccaacttgaagggcttctgctgtacaaatgtgggagaatttcatcgtcagaaggctctgcaaaggtctgaaagtcaccgaactctgtaagattctatcctgcttctattcctgtcaaaatataccagaaggaatggaactaccccctccaaaaaataaataaacaaacaaaccaccaaaccacgcacagacaaagcattcaatacacatgctaaaacataccactttagtttaaggactatagtgattccacactaggtaaggtgctttctgtaggcttttagttaatagttttgtcaagctaaagaagatctccagatggctaaacttttaaatcatgaatgaagtagatattaccaaattgctttttcagcatccatttagataatcatgttttttgcctttaatctgttaatgtagtgaattacagaaatacatttcctaaatcattacatcccccaaatcgttaatctgctaaagtacatctctggctcaaacaagactggttgtgacaggtttgtctctgtcagtttgtgactgttgggctggctcttcctacccctctgcttcttggtttggcctgaacattaattttattttatttttttaattttacctacaatcaatttcacaatgtgtgtt

SEQ ID NO: 6 - DNA, 460 bp, Mus musculus
ggctctgcaaaggtctgaaagtcaccgaactctgtaagattctatcctgcttctattcctgtcaaaatataccagaaggaatggaactaccccctccaaaaaataaataaacaaacaaaccaccaaaccacgcacagacaaagcattcaatacacatgctaaaacataccactttagtttaaggactatagtgattccacactaggtaaggtgctttctgtaggcttttagttaatagttttgtcaagctaaagaagatctccagatggctaaacttttaaatcatgaatgaagtagatattaccaaattgctttttcagcatccatttagataatcatgttttttgcctttaatctgttaatgtagtgaattacagaaatacatttcctaaatcattacatcccccaaatcgttaatctgctaaagtacatctctggctcaaacaagactggttgtgac

SEQ ID NO: 7 - DNA, 454 bp, Homo sapiens
ggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcgcgccccgagcgccttctccccaggaccccggtgtccctccccgcgccccgagcccgcgctctccttcccccgccctcagagcgctccccgcccctctgtctccccgcagcccgctagacgagccga

SEQ ID NO: 8 - DNA, 566 bp, Homo sapiens
ccaagaagaggacagaggcagaaagccagggacagagactgagaaacagagacctagaggcagaagaagactgagatagagatggacagagattgtgtcagacacagccccagagacagccagacagtctgagtcagacgcaaaccaaagacaagaaaacaggaaaacagacccagagattgggagagggaggggaaggagatgcggggagagccagcaccgccaccccccacactcaggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcg

SEQ ID NO: 9 - DNA, 454 bp, Mus musculus
agagagaagagagcccttcctccactctcaagctctggagggggtctctgccctcaccctcatccctccccagaatccttaaatcctctagactgtagctctgattttacagctgtcacagactcgtcctactagccagaggttggctcaggtaagcaccactggggaggtagcctagggtgcgctggggtgggtccagaggaagagctgcccagaactgtgggggaaggagcgggaccgaccatcaacagggggacttttcagggagaatgagagcaatcctctggaggcctgggagaggctgctgagttgctggtgcgcgagtcaccaacttttcctgcgctctcggtgtccggccagaatcccgaagtggcagctgagcacggggtggcagcttcgtccgccggctctcaaggcgtcccggtaacttcctttcccgcagtccaggagcaga

SEQ ID NO: 10 - DNA, 566 bp, Mus musculus
gaccgaccaggggagtccctggacttctttgttcctcttctcggggtggcgggactgattgtgtaaatctcttatctccaactttcactcttatctgtctctttaatcggcatattgaggatgagtggccaagcttattggtgttgctgggtcagacaatttaaaggcagtctaggggagaagcagacccagggagtcagagaggcagagagagaagagagcccttcctccactctcaagctctggagggggtctctgccctcaccctcatccctccccagaatccttaaatcctctagactgtagctctgattttacagctgtcacagactcgtcctactagccagaggttggctcaggtaagcaccactggggaggtagcctagggtgcgctggggtgggtccagaggaagagctgcccagaactgtgggggaaggagcgggaccgaccatcaacagggggacttttcagggagaatgagagcaatcctctggaggcctgggagaggctgctgagttgctggtgcgcgagtcaccaacttttcctgcgctctcggtgtccggcc

SEQ ID NO: 11 - DNA, 867 bp, Artificial Sequence
  Enhancer-Promoter Sequence
ctctgattttaaaggaagtagatacttcaaataattcatcatggagtgcaatattttctgtaggcttttagtagataacttcatcagtttaaagaagatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgacattgtatgtatttcagtttgcaaatattggactaggatttttgtatctatattccttagtttgacctgtaaattttatttcttgtactaagtattagcctcacgaaaggcattgtcaaatgttgctagcggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcgcgccccgagcgccttctccccaggaccccggtgtccctccccgcgccccgagcccgcgctctccttcccccgccctcagagcgctccccgcccctctgtctccccgcagcccgctagacgagccga

SEQ ID NO: 12 - DNA, 978 bp, Artificial Sequence
  Enhancer-Promoter Sequence
ctctgattttaaaggaagtagatacttcaaataattcatcatggagtgcaatattttctgtaggcttttagtagataacttcatcagtttaaagaagatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgacattgtatgtatttcagtttgcaaatattggactaggatttttgtatctatattccttagtttgacctgtaaattttatttcttgtactaagtattagcctcacgaaaggcattgtcaaatgttgctagccaagaagaggacagaggcagaaagccagggacagagactgagaaacagagacctagaggcagaagaagactgagatagagatggacagagattgtgtcagacacagccccagagacagccagacagtctgagtcagacgcaaaccaaagacaagaaaacaggaaaacagacccagagattgggagagggaggggaaggagatgcggggagagccagcaccgccaccccccacactcaggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcg

SEQ ID NO: 13 - DNA, 1243 bp, Artificial Sequence
  Enhancer-Promoter Sequence
gggtctccaacttgccaactgtagatcttggaacctttcatccttcataactgcataagccaattccttctaataaatctgtataatatatctgtctatataataaatatgtacttacataactctgtatgttacatctatctattctatctatctatctatctatctatctatctatctatctatcatctatctatctatttatctattctctgtgtctttggagaaccctgacatagtaagcaatcatatcacctgcaaatgatgaaagctgtgtattttccaaatcagtcgttttatgtctttttttcttgcactgactagtgccccctagagggaatgataattggaattattgtcttgctctgattttaaaggaagtagatacttcaaataattcatcatggagtgcaatattttctgtaggcttttagtagataacttcatcagtttaaagaagatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgacattgtatgtatttcagtttgcaaatattggactaggatttttgtatctatattccttagtttgacctgtaaattttatttcttgtactaagtattagcctcacgaaaggcattgtcaaatgttcaattgatataatgctagcggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcgcgccccgagcgccttctccccaggaccccggtgtccctccccgcgccccgagcccgcgctctccttcccccgccctcagagcgctccccgcccctctgtctccccgcagcccgctagacgagccga

SEQ ID NO: 14 - DNA, 1354 bp, Artificial Sequence
  Enhancer-Promoter Sequence
gggtctccaacttgccaactgtagatcttggaacctttcatccttcataactgcataagccaattccttctaataaatctgtataatatatctgtctatataataaatatgtacttacataactctgtatgttacatctatctattctatctatctatctatctatctatctatctatctatctatcatctatctatctatttatctattctctgtgtctttggagaaccctgacatagtaagcaatcatatcacctgcaaatgatgaaagctgtgtattttccaaatcagtcgttttatgtctttttttcttgcactgactagtgccccctagagggaatgataattggaattattgtcttgctctgattttaaaggaagtagatacttcaaataattcatcatggagtgcaatattttctgtaggcttttagtagataacttcatcagtttaaagaagatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgacattgtatgtatttcagtttgcaaatattggactaggatttttgtatctatattccttagtttgacctgtaaattttatttcttgtactaagtattagcctcacgaaaggcattgtcaaatgttcaattgatataatgctagccaagaagaggacagaggcagaaagccagggacagagactgagaaacagagacctagaggcagaagaagactgagatagagatggacagagattgtgtcagacacagccccagagacagccagacagtctgagtcagacgcaaaccaaagacaagaaaacaggaaaacagacccagagattgggagagggaggggaaggagatgcggggagagccagcaccgccaccccccacactcaggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcg

SEQ ID NO: 15 - DNA, 917 bp, Artificial Sequence
  Enhancer-Promoter Sequence
atctattctctgtgtctttggagaaccctgacatagtaagcaatcatatcacctgcaaatgatgaaagctgtgtattttccaaatcagtcgttttatgtctttttttcttgcactgactagtgccccctagagggaatgataattggaattattgtcttgctctgattttaaaggaagtagatacttcaaataattcatcatggagtgcaatattttctgtaggcttttagtagataacttcatcagtttaaagaagatccttagattatgaaacatttacaattatgaatgaatattagatgttatcaaatgctttttctgcatccatttagataatcatgtttttcctttaatctgttaatgcggtgaattacattaatagatttcctaagtcattaatctgctaaagtgcatttctgggacaaaccagacttggttatgaccaattgatataatgctagcggaggggtctccaccctcggagcggtctctcatccctccctagaatccttaaatcctctctcgctcagggcctcggccgcatctgtcacagacttgtcctgaaccgacagcggctggcgcaggtgactggcttggggcgggagcctgggtgtgcgctggggatggaccccgaggaagaggggccaagctgtcgggaagcggcagggctggaggggtggaggcagtggtcgggcgggaccccgggcgacagggttcggcgcttgtaagagcgagacggaggcccgggcaggccggctgagctaactccccagagccgaagtggaaggcgcgccccgagcgccttctccccaggaccccggtgtccctccccgcgccccgagcccgcgctctccttcccccgccctcagagcgctccccgcccctctgtctccccgcagcccgctagacgagccga

SEQ ID NO: 16 - DNA, 1257 bp, Artificial Sequence
  Chimeric Receptor
atggcccaaaggcttacaggtgaacagacactggaccactatgaggatagcacccatgcaagcatcttcacctataccaacagcaacagcaccaaaggtccctttgaaggccccaattatcacattgctcccaggtgggtgtaccacctcaccagcacctggatgattcttgtggtcgttgcatctgtcttcactaatggacttgtgctggcagccaccatgagattcaagaagctgcgccatccactgaactggattctggtgaacttggcagttgctgacctagcagagaccattattgccagcactatcagtgttgtgaaccaaatctatggctacttcgttctgggacaccctctgtgtgtcattgaaggctacattgtctcattgtgtggaatcacaggcctctggtccctggccatcatttcctgggagagatggctggtggtctgcaagccctttggcaatgtgagatttgatgctaagctggccactgtgggaatcgtcttctcctgggtctgggctgctatatggacggccccaccaatctttggttggagcaggtactggccttatggcctgaagacatcctgtggcccagacgtgttcagcggtacctcgtaccccggggttcagtcttatatgatggtcctcatggtcacgtgctgcatcttcccactcagcatcatcgtgctctgctacctccaagtgtggctggccatccgagcagtggcaaagcaacagaaagaatctgagtccactcagaaggccgagaaggaggtgacacgcatggtggtggtgatggtcttcgcatactgcctctgctggggaccctatactttctttgcatgctttgctactgcccaccctggctatgccttccaccctcttgtggcctccctaccatcctactttgccaaaagtgccactatctacaaccccattatctatgtctttatgaaccggcagtttcgaaactgcatcttacatctctttggaaagaaggttgatgatagctctgaactttccagcacctccaagacagaagtctcatctgtctcttcagtgtcacctgcagagcagaacgtgcagaagcggaagcgcagcctcaagaagacctccacgatggcggccccgcccaagagcgagaactcagaggacgccaagacagagaccagccaagtggcgcctgccaagagcaggatcaccagcgagggcgagtacatccccctggaccagatcgacatcaacgtgtaa

SEQ ID NO: 17 - DNA, 585 bp, Artificial Sequence
  IRES2 sequence
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca

SEQ ID NO: 18 - DNA, 726 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtccggataa

SEQ ID NO: 19 - DNA, 708 bp, Entacmaea quadricolor
atggtgggtgaggatagcgtgctgatcaccgagaacatgcacatgaaactgtacatggagggcaccgtgaacgaccaccacttcaagtgcacatccgagggcgaaggcaagccctacgagggcacccagaccatgaagatcaaggtggtcgagggcggccctctccccttcgccttcgacatcctggctaccagcttcatgtacggcagcaaaacctttatcaaccacacccagggcatccccgacttctttaagcagtccttccctgagggcttcacatgggagaggatcaccacatacgaagacgggggcgtgctgaccgctacccaggacaccagcctccagaacggctgcctcatctacaacgtcaagatcaacggggtgaacttcccatccaacggccctgtgatgcagaagaaaacactcggctgggaggccagcaccgagatgctgtaccccgctgacagcggcctgagaggccatagccagatggccctgaagctcgtgggcgggggctacctgcactgctccctcaagaccacatacagatccaagaaacccgctaagaacctcaagatgcccggcttctacttcgtggacaggagactggaaagaatcaaggaggccgacaaagagacctacgtcgagcagcacgagatggctgtggccaggtactgcgacctgcctagcaaactggggcacagctga

SEQ ID NO: 20 - DNA, 542 bp, Artificial Sequence
  WPRE sequence
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 21 - DNA, 215 bp, Artificial Sequence
  BGH pA signal
gcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 22 - DNA, 17 bp, Artificial Sequence
  sNRP-1 pA sequence
aaataaaatacgaaatg

SEQ ID NO: 23 - protein, 735 aa, adeno-associated virus 2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEFSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 24 - RNA, 13 nt, Artificial Sequence
  Kozak Sequence
gccgccaccaugg

SEQ ID NO: 25 - RNA, 13 nt, Artificial Sequence
  Kozak Sequence
gccgccgccaugg

(19) United States
(12) Patent Application Publication
(10) Pub. No.: US 2022/0387629 A1
(43) Pub. Date: Dec. 8, 2022

(54) ON-BIPOLAR CELL-SPECIFIC PROMOTERS FOR OCULAR GENE DELIVERY

(71) Applicant: UNIVERSITÄT BERN, Bern (CH)

(72) Inventors: Sonja KLEINLOGEL, Bern (CH); Elmar Carlos HULLIGER, Köniz (CH)

(21) Appl. No.: 17/755,745

(22) PCT Filed: Nov. 18, 2020
(86) PCT No.: PCT/EP2020/082588

§ 371 Date: May 6, 2022

(30) Foreign Application Priority Data
Nov. 18, 2019 (EP) 19209841.6

(51) Int. Cl. A61K 48/00; C12N 15/86; C12N 15/90; A61P 27/02

(52) U.S. Cl. CPC A61K 48/0066; C12N 15/86; C12N 15/907; A61K 48/0025; A61K 48/0058; A61K 48/0075; A61P 27/02; C12N 2750/14122; C12N 2750/14123; C12N 2750/14142; C12N 2830/008; C12N 2840/203

(57) ABSTRACT

The present invention relates to synthetic retinal ON-bipolar cell-specific promoter sequences and their use in therapeutic transgene delivery to the eye for the improvement and/or restoration of vision. The invention features metabotropic glutamate receptor 6 (mGluR6) promoters for an increased and more specific expression in ON-bipolar cells, in particular in cone ON-bipolar cells of the human macula.

The present invention relates to synthetic retinal ON-bipolar cell-specific promoter sequences and their use in therapeutic transgene delivery to the eye for the improvement and/or restoration of vision. The invention features metabotropic glutamate receptor 6 (mGluR6) promoters for an increased and more specific expression in ON-bipolar cells. In particular, in efficient expression in cone ON-bipolar cells exclusively present in the human macula.

### DESCRIPTION

### Background

Many causes for blindness have only limited treatment possibilities or no cure at all. Most prevalent among them are age-related macular degeneration (AMD) and inherited retinal diseases (IRDs) like retinitis pigmentosa (RP). These degenerative diseases are characterized by the progressive loss of photoreceptors (PRs) which eventually leads to complete blindness. Gene therapies, delivering curative DNA or RNA, replacing or silencing defect genes or encoding an exogenous curative gene are aimed at slowing disease progression, ameliorating symptoms or introducing lost function.

Optogenetic gene therapy is one of the most promising emerging technologies which could be employed for the treatment of blindness caused by retinal degeneration. Ongoing clinical trials of optogenetic therapies unspecifically target retinal ganglion cells (RGCs) with channelrhodopsins to reintroduce light sensitivity to the retina. In the future, next-generation cell-tailored optogenetic gene therapies will prove superior to these unspecific therapies. These next-generation therapies employ cell type-specific promoters to deliver novel and effective optogenetic tools to specific cell types of the retina. Most promising among cell type targets are retinal bipolar cells (BCs), the first interneurons of the retina that naturally receive direct input from the PRs. BCs are divided into ON- and OFF-type BCs, responding to either light increments or decrements, respectively, and expressing either mGluR6 or AMPA/Kainate glutamate receptors. ON-bipolar cells (OBCs) are particularly interesting targets for gene therapy. Mutations in OBC specific genes such as NYX, GRM6, GPR179 or TRPM1 all lead to complete blindness (congenital stationary night blindness) since these genes are involved in the mGluR6 signaling cascade and OBCs consequently become non-functional. More recently, expression of optogenetic proteins in OBCs has proven to restore vision in photoreceptor degenerative mouse models suffering from late stages of degeneration. Channelrhodopsin-2 (Lagali et al., Nat Neurosci 2008. 11:p. 667-675), rhodopsin (Cehajic-Kapetanovic et al., Curr Biol 2015. 25: p. 2111-2122) and chimeric Opto-mGluR6 (van Wyk et al., PLoS Biol 2015. 13: p. e1002143) have been successfully expressed in the OBCs of blind mice and restored functional vision at the retinal, cortical and behavioural levels. For all-above mentioned approaches the OBC type needs to be targeted specifically, in particular in the case of optogenetic approaches to avoid controversial signaling from off-target cells corrupting the retinal code. In addition, specific OBC targeting also allows for lower and thus safer AAV dosing. The lack of a functional and OBC-specific promoter hitherto prevented the clinical application of OBC-targeted gene therapies.

Short enhancer promoter sequences were typically employed in the field to achieve OBC-specific targeting in combination with an AAV-based gene therapy. This, since the packaging capacity of an AAV is limited to 4.7 kb and does typically not accommodate endogenous promoters of several kb in length. In this respect, enhancer promoter sequences derived from the OBC-specific mGluR6 glutamate receptor, exclusively expressed in the OBCs of the retina, have proven most successful. Until recently, a 200 bp long enhancer sequence derived from the murine Grm6 gene and in combination with an SV40 viral core promoter (Kim et al. J Neurosci, 2008. 28: p. 7748-7764.), abbreviated as 200En-SV40, was standardly used. However, the inventors recently showed that a variant thereof, 4x200En-SV40, which carries the enhancer sequence in quadruple (Cronin et al. EMBO Mol Med 2014. 6: p. 1175-1190) is neither OBC-specific nor functional in advanced degenerated retina (van Wyk et al. Front Neurosci 2017. 11: p. 161). More recently, an entirely murine Grm6 gene based short enhancer/promoter was designed (200En-mGluR500P) that expresses in the wild type C57BLJ6 mouse retina with relatively good OBC specificity (Lu et al. Gene Ther, 2016. 23: p. 680-9.). Nonetheless, expression in OBCs of the degenerated retina was not shown and expression was almost exclusively driven in the rod-type OBC. Therefore, the cone-type OBCs found exclusively in the macula of the retina of foveated animals—including humans—and connecting to foveal cones mediating high-acuity colour vision are virtually not targeted by 200En-mGluR500P, rendering this promoter not suited for restoration of high-acuity central human vision. In addition, a human GRM6 gene based promoter is favourable since it will be fully controlled by the human transcriptomic machinery, regulating gene expression—i.e. expression of protein levels mediating function but devoid of inducing cytotoxicity.

Based on the above-mentioned state of the art, the objective of the present invention is to provide means and methods to provide novel synthetic OBC-specific human promoters. This objective is attained by the subject-matter of the independent claims of the present specification.

### SUMMARY OF THE INVENTION

A first aspect of the invention relates to an isolated nucleic acid molecule of 850 base pairs (bp) to 1500 bp length comprising
a. an enhancer sequence element selected from SEQ ID NO 1 to 6, and
b. a promoter sequence element selected from SEQ ID NO 7 to 10.

An alternative of the first aspect of the invention relates to an isolated nucleic acid molecule of 850 base pairs (bp) to 1500 bp length comprising
a. an enhancer sequence element being at least (≥)70%, particularly ≥75%, more particularly ≥80%, more particularly ≥85%, more particularly ≥90%, more particularly ≥95%, even more particularly ≥98%, most particularly 100% identical to a sequence selected from SEQ ID NO 1 and 2; and
b. a promoter sequence element being ≥70%, particularly ≥75%, more particularly ≥80%, more particularly ≥85%, more particularly ≥90%, more particularly ≥95%, even more particularly ≥98%, most particularly 100% identical to a sequence of SEQ ID NO 7;
and said isolated nucleic acid molecule has ≥40%, particularly ≥50%, more particularly ≥60%, even more particularly ≥70%, more particularly ≥80%, even more particularly ≥90%, most particularly 100% of the cone ON bipolar cell-specificity from a sequence of SEQ ID NO 13 and a cone ON bipolar cell preference of ≥20%, particularly ≥25%, more particularly ≥30%, even more particularly ≥35%, more particularly ≥40%, most particularly ≥50%.

A second aspect of the invention relates to a nucleic acid expression vector comprising a nucleic acid molecule according to the first aspect.

A third aspect of the invention relates to the transgene driven by the promoter.

A fourth aspect of the invention relates to an adeno-associated virion particle comprising the isolated nucleic acid molecule according to the first aspect, the nucleic acid expression vector according to the second aspect or the transgene according to the third aspect.

A fifth aspect of the invention relates to an agent selected from the isolated nucleic acid molecule according to the first aspect, the nucleic acid expression vector according to the second aspect, the transgene according to the third aspect and the adeno-associated virion particle according to the fourth aspect for use as a medicament.

Administration forms comprising the agents of the invention are further aspects of the invention.

### Terms and Definitions

The term OBC in the context of the present specification relates to ON-bipolar cell.

The term RBC in the context of the present specifications relates to rod bipolar cell.

The term cOBC in the context of the present specifications relates to cone ON-bipolar cell.

The term RGC in the context of the present specification relates to retinal ganglion cell.

The term PR in the context of the present specification relates to photoreceptor.

The abbreviation AAV in the context of the present specification relates to adeno-associated virus. Except otherwise stated, AAV refers to all subtypes or serotypes and both replication-competent and recombinant forms.

The terms AAV virion and AAV viral particle in the context of the present specification relate to a viral particle composed of at least one AAV capsid protein and an encapsidated nucleic acid. Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.) and chemical methods.

The term AAV capsid in the context of the present specification relates to synthetic capsid (cap) genes. The AAV capsid disclosed herein may be used to package recombinant adeno-associated viruses for gene therapy.

The term homologous in the context of the present specification relates to sequences sharing a large part of their sequence, but differ in some positions by insertion, deletion or substitution of nucleic acids or amino acids.

The term transgene in the context of the present specification relates to a gene or genetic material that has been transferred from one organism to another. In the present context, the term may also refer to transfer of the natural or physiologically intact variant of a genetic sequence into tissue of a patient where it is missing. It may further refer to transfer of a natural encoded sequence the expression of which is driven by a promoter absent or silenced in the targeted tissue. The term transgene as used herein refers to a polynucleotide encoding a polypeptide of interest, which, when expressed in the damaged or diseased retina may be useful for improving or restoring vision. Transgenes of particular interest for restoration of photosensitivity or vision include photosensitive proteins, such as opsin genes, i.e. Channelrhodopsins, vertebrate opsins and variants thereof.

The term recombinant in the context of the present specification relates to a nucleic acid, which is the product of one or several steps of cloning, restriction and/or ligation and which is different from the naturally occurring nucleic acid. A recombinant virus particle comprises a recombinant nucleic acid.

The term intravitreal administration in the context of the present specification relates to a route of administration of a pharmaceutical agent, for example a virus, in which the agent is delivered into the vitreous body of the eye. Intravitreal administration is a procedure to place a medication directly into the space in the back of the eye called the vitreous cavity, which is filled with a jelly-like fluid called the vitreous humour gel.

The term subretinal administration in the context of the present specification relates to a route of administration of a pharmaceutical agent, particularly a virus in the context of this specification, into the space between retinal pigment epithelium (RPE) cells and photoreceptors.

“Nucleotides” in the context of the present specification are nucleic acid or nucleic acid analogue building blocks, oligomers of which are capable of forming selective hybrids with RNA or DNA oligomers on the basis of base pairing. The term nucleotides in this context includes the classic ribonucleotide building blocks adenosine, guanosine, uridine (and ribosylthymine), cytidine, the classic deoxyribonucleotides deoxyadenosine, deoxyguanosine, thymidine, deoxyuridine and deoxycytidine.

In the context of the present specifications the terms sequence identity and percentage of sequence identity refer to the values determined by comparing two aligned sequences. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless stated otherwise, sequence identity values provided herein refer to the value obtained using the BLAST suite of programs (Altschul et al., J. Mol. Biol. 215:403-410 (1990)) using the above identified default parameters for protein and nucleic acid comparison, respectively.

In the context of the present specification, the term upstream refers to a direction towards the 5′ end. For enhancer and promoter sequences, single-stranded sequences are given in this application and when the enhancer is upstream of the promoter, this means that the enhancer is in 5′-direction of the promoter. Analogously, the term downstream refers to a direction towards the 3′ end.

In the context of the present specification, the term spacer sequence refers to a nucleic acid of variable length that is used to connect the enhancer and the promoter in order to generate a single chain nucleic acid molecule. Exemplary embodiments of linkers useful for practicing the invention specified herein are oligo nucleic acid chains consisting of 1 to 1000 nucleic acids.

The cone ON bipolar cell (cOBC)-specificity in human retinal explants is measured using the following protocol.

First, the promoter is combined with the reporter transgene mCitrine and packaged into the self-complementary (sc) AAV vector scAAV2(7m8) (Dalkara et al. Sci Transl Med 2013. 5: p. 189ra76). Approximately 10¹⁰ vg (vector genomes) are added to the RGC side of cultured post-mortem human retinal explants at day 0 as described in detail in (van Wyk et al. Front Neurosci 2017. 11: p.161). Retinas are fixed at day 7 of culture with 4% PFA and subsequently cryoprotected (10/20/30% sucrose in PBS) and frozen. Retinal cryosections are triple-stained with antibodies against the transgene mCitrine (Invitrogen, A11122, 1:500), the ubiquitous OBC marker Gαo (EMD, MAB3073, 1:750) and the RBC specific antibody PKCα (Santa Cruz, sc8393, 1:750). Expressing RBCs are identified as [mCitrine(+), PKCα(+)], whereas [mCitrine(+), PKCα(−), Gαo(+)] cells are identified as expressing cOBCs. cOBC type specificity is determined by the ratio of expressing cOBCs of all expressing OBCs:

$$[ \frac{N \{ m C i t r i n e ( + ) , PKC α ( - ) , G α o ( + ) \}}{N \{ m C i t r i n e ( + ) , G α o ( + ) \}} ] \qquad ( 1 )$$

With N being the number of cells with the staining characteristics given in brackets.

The cone ON bipolar cell preference is subsequently determined as follows.

The amount of RBCs and cOBCs is not identical and varies in different retinal regions. The explants are produced from the mid-periphery of the retina where the ratio of RBCs to cOBCs

$$[ \frac{N \{ PKC α ( + ) \}}{N \{ PKC α ( - ) , G α o ( + ) \}} ] \qquad ( 3 )$$

is approximately constant over the small area of the explant. Consequently, the ratio of expressing cOBCs to expressing RBCs

$$[ \frac{N \{ m C i t r i n e ( + ) , PKC α ( - ) , G α o ( + ) \}}{N \{ m C i t r i n e ( + ) , PKC α ( + ) , G α o ( + ) \}} ] \qquad ( 4 )$$

can be assumed to be constant in the explant as well. This allows the calculation of the cOBC over RBCs preference ratio factor

$$x = \frac{N \{ PKC α ( + ) \} * N \{ m C i t r i n e ( + ) , PKC α ( - ) , G α o ( + ) \}}{N \{ PKC α ( - ) , G α o ( + ) \} * N \{ m C i t r i n e ( + ) , PKC α ( + ) , G α o ( + ) \}} \qquad ( 5 )$$

which accounts for the specific distribution of cOBCs and RBCs in the explant, by multiplying (3) with (4). The cOBC preference in percent is then calculated with

$$\frac{100 \% * x}{1 + x} \qquad ( 6 )$$

As used herein, the term treating or treatment of any disease or disorder (e.g. loss of vision) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to introducing an exogenous, therapeutic function into a target cell type. Methods for assessing treatment and/or prevention of disease are generally known in the art, unless specifically described herein below.

### Detailed Description of the Invention

This invention discloses human GRM6 enhancer promoter sequences with enhanced OBC-specificity and far enhanced cOBC-induced protein expression compared to 200En-mGluR500P, in mouse and human post-mortem retina. The promoters described herein consist of a modified metabotropic glutamate receptor 6 (mGluR6) promoter that contains sequences from regulatory elements that direct the expression of the mGluR6 protein to OBCs, in particular RBCs and cOBCs. The invention features an isolated nucleic acid molecule or a nucleic acid expression vector comprising an mGluR6 enhancer or a variant thereof and an mGluR6 promoter or a variant thereof. This novel GRM6 enhancer promoter sequence drives efficient transgene expression for the first time in cOBCs of the human retina, in particular of the human parafovea. Further, the novel human GRM6 enhancer promoter sequences in combination with an optogene (MWOPN_mGluR6, SEQ ID NO: 16) led to widespread OBC-specific expression in the degenerated murine (rd1, C3HHe/OuJ) retina and restored functional vision (optomoter response) in otherwise blind, photoreceptor degenerated mice. The novel human GRM6 enhancer/promoter showed highly efficient, widespread and specific OBC targeting in mouse and human retina.

A first aspect of the invention relates to an isolated nucleic acid molecule comprising
a. an enhancer sequence element selected from SEQ ID NO 1 to 6, and
b. a promoter sequence element selected from SEQ ID NO 7 to 10.

An alternative of the first aspect of the invention relates to an isolated nucleic acid molecule of 850 base pairs (bp) to 1500 bp length comprising
a. an enhancer sequence element selected from SEQ ID NO 1 to 6, and
   a promoter sequence element selected from SEQ ID NO 7 to 10.

Another alternative of the first aspect of the invention relates to an isolated nucleic acid molecule comprising
a. an enhancer sequence element being ≥70%, particularly ≥75%, more particularly ≥80%, more particularly ≥85%, more particularly ≥90%, more particularly ≥95%, even more particularly ≥98%, most particularly 100% identical to a sequence selected from SEQ ID NO 1 and 2; and
b. a promoter sequence element being ≥70%, particularly ≥75%, more particularly ≥80%, more particularly ≥85%, more particularly ≥90%, more particularly ≥95%, even more particularly ≥98%, most particularly 100% identical to a sequence of SEQ ID NO 7;
and said isolated nucleic acid molecule has ≥40%, particularly ≥50%, more particularly ≥60%, even more particularly ≥70%, more particularly ≥80%, even more particularly ≥90%, most particularly 100% of the cone ON bipolar cell-specificity from a sequence of SEQ ID NO 13 and a cone ON bipolar cell preference of ≥20%, particularly ≥25%, more particularly ≥30%, even more particularly ≥35%, more particularly ≥40%, most particularly ≥50%.

Another alternative of the first aspect of the invention relates to an isolated nucleic acid molecule of 850 base pairs (bp) to 1500 bp length comprising
a. an enhancer sequence element being ≥70%, particularly ≥75%, more particularly ≥80%, more particularly ≥85%, more particularly ≥90%, more particularly ≥95%, even more particularly ≥98%, most particularly 100% identical to a sequence selected from SEQ ID NO 1 and 2; and
b. a promoter sequence element being ≥70%, particularly ≥75%, more particularly ≥80%, more particularly ≥85%, more particularly ≥90%, more particularly ≥95%, even more particularly ≥98%, most particularly 100% identical to a sequence of SEQ ID NO 7;
and said isolated nucleic acid molecule has ≥40%, particularly ≥50%, more particularly ≥60%, even more particularly ≥70%, more particularly ≥80%, even more particularly ≥90%, most particularly 100% of the cone ON bipolar cell-specificity from a sequence of SEQ ID NO 13 and a cone ON bipolar cell preference of ≥20%, particularly ≥25%, more particularly ≥30%, even more particularly ≥35%, more particularly ≥40%, most particularly ≥50%.

The cOBC specificity and the cOBC expression level is measured as described above.

The inventors have shown that a combination of SEQ ID NO 1 or 2 with SEQ ID NO 7 results in a high cone ON bipolar cell-specificity and a high cone ON bipolar cell expression level. The skilled person in the art is able to find similar sequences with equal cone ON bipolar cell-specificity and cone ON bipolar cell expression level based on the disclosure of this invention.

In certain embodiments, the enhancer sequence element is upstream of the promoter sequence element.

In certain embodiments, the isolated nucleic acid molecule additionally comprises a spacer sequence of length 1 to 1000 basepairs, particularly 1 to 394 basepairs. In certain embodiments, the spacer is located between the enhancer and the promoter. In certain embodiments, the isolated nucleic acid molecule additionally comprises a spacer sequence of length 1 to 1000 basepairs, particularly 1 to 394 basepairs, and the spacer is located between the enhancer and the promoter.

In certain embodiments, the isolated nucleic acid molecule comprises a sequence selected from SEQ ID NO 11-SEQ ID NO 15.

In certain embodiments, the isolated nucleic acid molecule comprises the sequence SEQ ID NO 11 or SEQ ID NO 13.

A second aspect of the invention relates to a nucleic acid expression vector comprising a nucleic acid molecule according to the first aspect.

In certain embodiments, the viral vector is a viral genome.

In certain embodiments, the vector is an adeno-associated virus vector or a recombinant adeno-associated vector (rAAV).

In certain embodiments, the AAV vector is either a single-stranded vector (ssAAV) or a self-complementary vector (scAAV).

In certain embodiments, the vector is a recombinant AAV1, AAV2, AAV3, AAV4, AAV5, AAV6, AAV7, AAV8, AAV9, AAV10, AAV11, or AAV12 vector. In certain embodiments, the vector is a recombinant AAV2 vector.

In certain embodiments, the nucleic acid expression vector additionally comprises
a. a sequence encoding a capsid protein, and
b. a transgene.

From 5′-end to 3′-end, the isolated nucleic acid molecule comprises first the enhancer, then optionally the spacer and then the promoter. The transgene is located in 3′-direction of the promoter. In certain embodiments, the transgene is preceded by an optimized KOZAK sequence.

The KOZAK sequence has the consensus (gcc)gccAccAUGG (SEQ ID NO 24) or (gcc)gccGccAUGG (SEQ ID NO 25) and is important in the initiation of the translation.

In certain embodiments, the nucleic acid expression vector also comprises a WPRE (Woodchuck hepatitis virus post-transcriptional regulatory element) regulatory sequence. The WPRE is a DNA sequence that, when transcribed, creates a tertiary structure enhancing expression. In certain embodiments, the nucleic acid expression vector also comprises a polyA tail, which is inserted downstream of the transgene. The polyA tail promotes translation of the transgene.

In certain embodiments, the capsid protein is AAV2, AAV2(7m8) or AAV8(BP2).

A third aspect of the invention relates to the transgene driven by the promoter.

In certain embodiments, the transgene is NYX, GRM6, GPR179 or TRPM1 to restore light sensitivity or vision in congenital stationary night blindness.

In certain embodiments, the transgene comprises or essentially consists of the sequence of SEQ ID NO 16.

In certain embodiments, the transgene is an opsin gene restoring light detection or vision.

In certain embodiments, the opsin gene is selected from the group consisting of channelrhodopsin, melanopsin, rhodopsin, cone opsins, pineal opsin, photopsins, halorhodopsin, bacteriorhodopsin, proteorhodopsin, jellyfish opsin, jumping spider opsin or any functional variant or fragment thereof.

In certain embodiments, the opsin gene is a chimeric protein between an opsin and the metabotropic glutamate receptor mGluR6 of retinal OBCs.

In certain embodiments, the chimeric protein is Opto-mGluR6.

In certain embodiments, the chimeric protein is murine or human MWOPN_mGluR6 (SEQ ID NO: 16).

A fourth aspect of the invention relates to an adeno-associated virion particle comprising the isolated nucleic acid molecule according to the first aspect or the nucleic acid expression vector according to the second aspect.

A fifth aspect of the invention relates to an agent selected from the isolated nucleic acid molecule according to the first aspect or the nucleic acid expression vector according to the second aspect, and the adeno-associated virion particle according to the third and fourth aspects for use as a medicament.

A further aspect relates to an agent selected from the isolated nucleic acid molecule according to the first aspect, the nucleic acid expression vector according to the second aspect, the transgene according to the third aspect and the adeno-associated virion particle according to the fourth aspect for use in treatment of a condition affecting a retinal bipolar cell.

A further aspect relates to an agent selected from the isolated nucleic acid molecule according to the first aspect, the nucleic acid expression vector according to the second aspect, the transgene according to the third aspect and the adeno-associated virion particle according to the fourth aspect for use in treatment of congenital stationary night blindness or rod-cone and cone-rod dystrophies, in particular of retinitis pigmentosa and macular degeneration.

A further aspect relates to an agent selected from the isolated nucleic acid molecule according to the first aspect, the nucleic acid expression vector according to the second aspect, the transgene according to the third aspect and the adeno-associated virion particle according to the fourth aspect, wherein the agent is administered by
a. intravitreal administration, particularly by intravitreal injection, or by
b. subretinal injection.

A further aspect relates to a method of treatment administering the agent of the invention to a patient in need thereof.

Wherever alternatives for single separable features such as, for example, a promoter sequence or medical indication are laid out herein as “embodiments”, it is to be understood that such alternatives may be combined freely to form discrete embodiments of the invention disclosed herein. Thus, any of the alternative embodiments for a promoter sequence may be combined with any medical indication compromising OBC function and any DNA delivery vehicle or method, including alternative viruses, nanoparticles, liposomes or “naked” DNA delivery by using, for example, a gene gun or electroporation.

A non-limiting list of retinal diseases that may benefit from the methods described herein include congenital night blindness, macular degeneration, age-related macular degeneration, congenital cone dystrophies and a large group of retinitis pigmentosa (RP)-related disorders.

### Items

1. An isolated nucleic acid molecule of 850 base pairs (bp) to 1500 bp length, comprising
   a. an enhancer sequence element selected from SEQ ID NO 1 to 6, and
   b. a promoter sequence element selected from SEQ ID NO 7 to 10.
2. An isolated nucleic acid molecule of 850 base pairs (bp) to 1500 bp length, comprising
   a. an enhancer sequence element being at least (≥)70%, particularly ≥75%, ≥80%, more particularly ≥85%, more particularly ≥90%, more particularly ≥95%, even more particularly ≥98%, most particularly 100% identical to a sequence selected from SEQ ID NO 1 and 2; and
   b. a promoter sequence element being 70%, particularly ≥75%, more particularly ≥80%, more particularly ≥85%, more particularly ≥90%, more particularly ≥95%, even more particularly ≥98%, most particularly 100% identical to a sequence of SEQ ID NO 7;
   and said isolated nucleic acid molecule has ≥40%, particularly ≥50%, more particularly ≥60%, even more particularly ≥70%, more particularly ≥80%, even more particularly ≥90%, most particularly 100% of the cone ON bipolar cell-specificity from a sequence of SEQ ID NO 13 and a cone ON bipolar cell preference of ≥20%, particularly ≥25%, more particularly ≥30%, even more particularly ≥35%, more particularly ≥40%, most particularly ≥50%.
3. The isolated nucleic acid molecule according to item 1 or 2, wherein the isolated molecule consists of one and only one of said enhancer sequence elements, one and only one of said promoter sequence elements and optionally, a spacer separating the enhancer sequence element from the promoter sequence element.
4. The isolated nucleic acid molecule according to any one of the preceding items comprising or consisting of a sequence selected from SEQ ID NO 11-SEQ ID NO 15, or comprising or consisting of a sequence characterized by ≥98% identity to a sequence selected from SEQ ID NO 11-SEQ ID NO 15.
5. The isolated nucleic acid molecule according to any one of the preceding items comprising or consisting of the sequence SEQ ID NO 11 or SEQ ID NO 13, or comprising or consisting of a sequence characterized by ≥98% identity to SEQ ID NO 11 or SEQ ID NO 13, particularly comprising or consisting of the sequence SEQ ID NO 13, or comprising or consisting of a sequence characterized by ≥98% identity to SEQ ID NO 13.
6. A nucleic acid expression vector comprising a nucleic acid molecule according to any one of the previous items.
7. The nucleic acid expression vector according to item 6, wherein the nucleic acid expression vector is an adeno-associated virus vector or a recombinant adeno-associated vector (rAAV), particularly wherein the nucleic acid expression vector is a recombinant AAV1, AAV2, AAV3, AAV4, AAV5, AAV6, AAV7, AAV8, AAV9, AAV10, AAV11, or AAV12 vector, more particularly wherein the nucleic acid expression vector is a recombinant AAV2 vector.
8. The nucleic acid expression vector according to any one of items 6 to 7, additionally comprising
   a. a sequence encoding a capsid protein, and
   b. a transgene.
9. The nucleic acid expression vector according to item 8, wherein the transgene comprises the sequence of SEQ ID NO 16.
10. An adeno-associated virion particle comprising the isolated nucleic acid molecule according to any one of items 1 to 5 or the nucleic acid expression vector according to any one of items 6 to 9.
11. An agent selected from the isolated nucleic acid molecule according to any one of items 1 to 5 or the nucleic acid expression vector according to any one of items 6 to 9, and the adeno-associated virion particle according to item 10 for use as a medicament.
12. An agent selected from the isolated nucleic acid molecule according to any one of items 1 to 5, the nucleic acid expression vector according to any one of items 6 to 9, and the adeno-associated virion particle according to item 10 for use in treatment of a condition affecting a retinal bipolar cell, particularly in treatment of congenital stationary night blindness (CSBN1) or rod-cone and cone-rod dystrophies, more particularly of retinitis pigmentosa and macular degeneration.
13. An agent selected from the isolated nucleic acid molecule according to any one of items 1 to 5, the nucleic acid expression vector according to any one of items 6 to 9, and the adeno-associated virion particle according to item 10, wherein the agent is administered by
   a. intravitreal administration, particularly by intravitreal injection, or by
   b. subretinal injection.

The invention is further illustrated by the following examples and figures, from which further embodiments and advantages can be drawn. These examples are meant to illustrate the invention but not to limit its scope.

### BRIEF DESCRIPTION OF THE FIGURES

FIG. 1 Genome browser view of the human GRM6 sequences chosen for promoter design. (A) Distal enhancer region of human GRM6 (positioned at approx. −14 kb rel. to translation start site (TLSS)), indicating the three selected enhancer elements including the 310 bp conserved region between murine Grm6 and human GRM6 (horizontally striped) as well as the 188 bp syntenic region to Kim's murine 200En(Grm6) (horizontally striped and shaded section). (B) Promoter sequence of human GRM6, including the transcription start site (TSS) and translation start site (TLSS), latter defined as position 0 by the inventors. The two promoters selected are also indicated and the 167 bp conserved region between mouse and human genes shown by the horizontally striped section. The graphs were downloaded from the UCSC Genome Browser at https://cienome.ucsc.edu/ and modified. Grey shaded stretches illustrate potentially relevant cis-regulatory regions, including transcription factor binding sites, interspecies conserved regions (Vert. Cons) and Dnase hypersensibility clusters (Dnase clusters). Additionally, the track for the H3K27Ac Mark signal peak and Chip-seq peaks were also considered.
FIG. 2 Promoter driven mCitrine expression strength in OBCs of post-mortem human retinal explants. Human retinal explants were transduced with scAAV2(7m8)-407En_566P(hGRM6)-mCitrine (n=3), scAAV2(7m8)-444En_454P(hGRM6)-mCitrine (n=3), scAAV2(7m8)-770En_454P(hGRM6)-mCitrine (n=5), scAAV2(7m8)-407En_454P(hGRM6)-mCitrine (n=3) and scAAV(7m8)-200En-mGluR500P-mCitrine (n=4). mCitrine was immunohistochemically labeled and the fluorescence intensity in expressing ON-bipolar cells determined as a measure of transgene expression strength. The 566P promoter element mediated much weaker mCitrine expression in OBCs compared to promoter combinations with proximal element 454P. 454P was therefore selected for all subsequent experiments. 770En_454P(hGRM6) (F=5.42±0.9; mean±s.d.) and 444En_454P(hGRM6) (F=5.59±0.51; mean±s.d.) performed equally well in terms of transgene expression strength and significantly better than the murine genome-derived 200En-mGluR500P (F=3.94±0.45; mean±s.d.). * represents P≤0.05, ** represents P≤0.01, *** represents P≤0.001 and n.s. represents non-significant differences (one-way ANOVA with Tukey's honest significance test).
FIG. 3 Promoter specificities for cone ON-bipolar cells (cOBCs) in human retinal explants. Human retinal explants were transduced with scAAV2(7m8)-444En_454P(hGRM6)-mCitrine, scAAV2(7m8)-770En_454P(hGRM6)-mCitrine, scAAV2(7m8)-407En_454P(hGRM6)-mCitrine or scAAV(7m8)-200En-mGluR500P-mCitrine. Vertical cryo-sections were labeled against mCitrine, PKCα (for rod bipolar cells (RBCs) and Gα0 (ubiquitous marker for OBCs) and mCitrine expressing cells were counted. (A) From cell counting, 770En_454P(hGRM6) drove expression in significantly more cOBCs than 200En-mGluR500P. (B) Normalizing the amount of targeted cOBCs and RBCs to their overall number within the particular retinal regions from where the counts were taken visualizes that 770En_454P(hGRM6) and 407En_454P(hGRM6) drive expression much more efficiently in cOBCs than 200En-mGluR500P, which has a clear preference for RBCs. 770En_454P(hGRM6) even shows equal preference for cOBCs and RBCs of 50% each, an important hallmark for foveal gene therapy where only cOBCs exist. (C) The cOBC type preference of promoter 444En_454P(hGRM6) (n=5) was not significantly different to that of promoter 770En_454P(hGRM6). Shown are mean±s.d., * represents P≤0.05, ** represents P≤0.01 (one-way ANOVA with Tukey's honest significance test). Only significant differences are indicated.
FIG. 4 OBC expression efficacy and specificity of 770En_454P(hGRM6)-driven mCitrine expression in human retinal explants. (A) Comparing 770En_454P(hGRM6)-driven mCitrine expression to 200En-mGluR500P driven mCitrine expression when packaged into scAAV2(7m8). 770En_454P(hGRM6) shows a much higher OBC preference compared to 200En-mGluR500P and latter has in addition a significantly higher off-target expression in amacrine cells. Shown are the % of expressing cells of a particular cell type as means±s.d., ** represents P≤0.01 and *** represents P≤0.001 (Student's T-Test).
FIG. 5 Promoter 770En_454P(hGRM6) drives transgene expression reliably and wide-spread in the degenerated rd1 mouse retina. rd1 mice were injected with 3×10⁹ vg of AAV carrying the transgene MWOPN_mGluR6_IRES2_TurboFP635 (SEQ ID NO: 16, plasmid map FIG. 9) at 22 weeks of age. The sketches in A-D represent the transduced retinal areas in retinal whole mounts in grey. A) ssAAV2(7m8) in combination with 200En-mGluR500P also expresses in the rd1 retina, but only in restricted areas (as opposed to 4xGrm6-SV40). B) ssAAV(7m8) in combination with 770En_454P(hGRM6) leads to more extensive and widespread transduction of the degenerated retina compared to 200En-mGluR500P, probably due to an increased expression strength which overcomes the threshold for expression when degeneration-caused Grm6 downregulation has occurred. C) Example laser scanning micrograph of a retinal whole-mount of a treated retina from a rd1 mouse undergoing OKR testing (see Example 7 and FIG. 6), where TurboFP635 was labelled immunocytochemically (depicted in sketch B). (D) Mean expression specificity (% expressing OBCs of all cells expressing, 66.6±8.5%) and efficiency (% of expressing OBCs of all OBCs, 54.7±8.3) in the rd1 degenerated retina. Mean±s.d., N=9 (Student's T-test).
FIG. 6 Vision restoration determined by the optomotor reflex in completely photoreceptor-less rd1 mice intravitreally and bilaterally treated with ssAAV(7m8)-MWOPN_mGluR6_IRES2_TurboFP635 at 22 weeks of age. Visual acuity was measured 41, 47, 55, 82 and 112 days after transduction by determining the threshold of spatial frequencies at which the optokinetic response was still elicited in a virtual optomotor system. The treated mice (n=3) showed a significant increase in visual acuity compared to non-injected control rd1 littermates (n=7) but still had significantly lower visual acuity than the wild type control mice (C57BLJ6J, n=10). Shown mean (over all trials and individuals)±s.d., ** represents P≤0.01 and *** represents P≤0.001 (Student's T-test).
FIG. 7 Highly efficient mCitrine expression in the cOBCs of an explanted human macula by promoter 770En_454P(hGRM6). Example explant of a human macula transduced with scAAV2(7m8)-770En_454P(hGRM6)-mCitrine and immunohistochemically labeled against the transgene mCitrine as well as for OBCs (Gαo) and cellular nuclei (DAPI). (A) is a sketch of the human macula and the areas from where the photomicrographs in B and C were taken. The foveola only contains M- and L-cone photoreceptors and neither OBCs or RGCs, since their cell bodies are pushed aside for the light to pass without diffraction to the photoreceptors. The fovea contains only cones (M, L and S) and is the area of highest acuity vision with the midget system, where each cone connects to one BPC and to one RGC. (B) shows a section through the parafovea, with DAPI (top micrograph) showing a clear layering of photoreceptors (ONL), BPCs and amacrine cells (INL) and the 3-dimenional layering of RGCs (GCL) indicative of the macula. The bottom micrograph shows exclusively the transgene labeling, indicative of mCitrine expression exclusively in the INL where the OBCs are located. (C) shows a section through the fovea. The photomicrograph on the left solely shows Gαo labeling of the OBCs, which are all devoid of PKC labeling (not shown here) and therefore clearly identified as cOBCs. The right photomicrograph depicts in addition mCitrine labeling within the cytoplasm, indicating that virtually each cOBC of the fovea is expressing mCitrine (arrowheads). This is a very clear evidence that 770En_454P(hGRM6) drives excellent expression in cOBCs, in particular cOBCs of the human macula, and is therefore well suited for high acuity vision restoration in human patients.
FIG. 8 Plasmid map of the AAV plasmid encoding a cone opsin and mGluR6 chimeric optogenetic protein, MWOPN_mGluR6-IRES2-TurboFP635, under the novel 770En_454P(hGRM6) promoter. TurboFP635 is a red fluorescent protein marker for identification of expression, WPRE and BGHpA are regulatory sequences and the 5′ and 3′ ITRs (internal repeats) the regions used by the AAV machinery to package the transgene (in between the ITRs) into the capsid. This plasmid was used for the transduction of rd1 degenerated mouse retinas (FIGS. and Examples 5 & 6). In-Fusion Primers for cloning are also given.
FIG. 9 Example of OBC expression specificity and efficacy of 770En_454P(hGRM6) and 444En_454P(hGRM6) driving mCitrine expression in human retinal explants. Vertical cryosections through human retinal explants transduced with scAAV2(7m8)-770En_454P(hGRM6)-mCitrine (A) and scAAV2(7m8)-444En_454P(hGRM6)-mCitrine (B), respectively. Cryosections were labelled with the nuclear stain DAPI (grey, shown only on the far left of the micrographs for orientation) and against the transgene mCitrine marker (white). 770En_454P(hGRM6) has an OBC efficacy (percent of bright cells in the INL) of 85.2%±12.3% (n=4) and 444En_454P(hGRM6) of 87.9%±6.5% (n=5). ONL outer nuclear layer, INL inner nuclear layer, GCL ganglion cell layer. The bipolar cells are located in the peripheral INL.

### EXAMPLES

### Example 1: Analysis of Grm6 Gene Expression Changes in the rd1 Mouse Model

The inventors selected the gene (Grm6 in mouse and GRM6 in human) encoding the metabotropic glutamate receptor 6 (mGluR6) selectively expressed in ON-bipolar cells (OBCs) of the retina as a template for promoter design. This was because mGluR6's expression is selective to OBCs, which was recently confirmed by a single-cell transcriptome analyses of adult mouse retina (Siegert et al. Nat Neurosci 2012. 15: p. 487-95) and also clearly obvious in a transgenic mouse line previously generated by the inventors where the full-length Grm6 promoter drives transgene expression specifically in retinal OBCs (van Wyk et al., PloS Biol 2015. 13: p. e1002143). Further, short promoter versions derived from the murine Grm6 gene have been successfully constructed and shown to drive preferential expression in OBCs (Cronin et al., EMBO Mol Med, 2014. 6(9): p. 1175-1190; Kim et al. J Neurosci 2008. 28: p. 7748-7764; Lagali et al. Nat Neurosci 2008. 11 p: 667-675). Kim et al. initially chose a distal 200 bp enhancer sequence in the promoter of the murine Grm6 gene, which enhanced OBC-specific expression in the wildtype mouse retina. This enhancer sequence was subsequently employed in the 4xGRM6-SV40 promoter [Cronin, T., et al., EMBO Mol Med, 2014. 6(9): p. 1175-1190], which contains four of these 200 bp enhancer sequences in tandem. However, the inventors showed recently that the 4xGRM6-SV40 promoter [Cronin, T., et al., EM BO Mol Med, 2014. 6(9): p. 1175-1190] was completely downregulated in the degenerated rd1 (C3H/HeOu) mouse retina, even when gene therapy was performed at 3.5 weeks of age before completed photoreceptor degeneration (van Wyk et al. Front Neurosci 2017. 11: p. 161). This makes 4xGRM6-SV40 (and equally GRM6-SV40) not suited to treat degenerated retina. In addition, the SV40 basal viral promoter is inflicted with issues such as silencing under chronic activation and protein overexpression leading to cellular cytotoxicity. In order to design better-suited OBC-specific promoters, the inventors first investigated if Grm6 expression remained upregulated during the degeneration process in the rd1 degeneration mouse model. The inventors employed the rd1 mouse model under the rationale that promoters active in this severe and rapid degeneration model are likely to be active in most, less severe degenerative diseases of the retina. This was previously exemplified by the inventors comparing transgene expression in the slower degenerating rd10 (B6.CXB1-Pde6brd10) mouse model where 4xGRM6-SV40 was able to still drive some expression [van Wyk, M., et al., Front Neurosci, 2017. 11(161): p. 161.]. The inventors quantified Grm6 gene expression in rd1 mouse retinas by real-time quantitative PCR at time points P14, P21, P28 and P54 and compared expression levels to wildtype C57BLJ6J mouse retinas. Ribosomal protein L8 (Rpl8) expression was used for normalization of expression levels. Grm6 expression remained constant during degeneration (P=0.8795), with the exception of a small downregulation (0.59-fold) between P21 and P28. From this, the inventors concluded that the severe downregulation observed for the 4xGRM6-SV40 promoter [van Wyk, M., et al., Front Neurosci, 2017. 11(161): p. 161.] was not likely due to a downregulation of Grm6 enhancer elements, but probably the consequence of a diminished functionality of the SV40 basal promoter with progressing degeneration. Consequently, the Grm6 gene was used by the inventors as a template for OBC-specific promoter design.

### Example 2: Design of GRM6-Based Promoters

To align with the human transcription machinery in light of a future use in a human therapy, the inventors employed the human GRM6 sequence and not the murine Grm6 sequence as a template.

The inventors used the Basic Local Alignment Search Tool (BLAST, https://blast.ncbi.nlm.nih.gov/Blast.cgi) optimized for “somewhat similar” sequences (blastn) [Altschul et al., J Mol Biol, 1990. 215(3): p. 403-10; Coordinators, Nucleic Acids Res, 2018. 46(D1): p. D8-D13] to align murine Grm6 and human GRM6 gene sequences. For enhancer specification, the inventors aligned 1500 bp around the murine 200En enhancer sequence identified by Kim et al. [Kim et al., J Neurosci, 2008. 28(31): p. 7748-64.]. The inventors found a 310 bp long conserved sequence betweem mouse and human genomes [−13819 to −13510 rel. to the translation start site (TLSS) of GRM6] extending beyond the 200En sequence defined by Kim et al. in both 3′ and 5′ direction (FIG. 1A, horizontally striped sections). To identify promoter sequences, the inventors focused on the GRM6 sequences 5′ of the translation start site (TLSS, defined as position 0 by the invetors) (FIG. 1B). After alignment, the inventors used the Genome Browser of the Genomics Institute of the University of California Santa Cruz (UCSC Genome Browser) [Church et al., PloS Biol, 2011. 9(7): p. e1001091.], [Kent et al., Genome Res, 2002. 12(6): p. 996-1006.; Kuhn et al. Brief Bioinform, 2013. 14(2): p. 144-61.] [https://genome.ucsc.edu/; genome assembly February 2009 (GRCh37/hg19] to identify potential regulatory sequences of the GRM6 gene, such as inter-species conserved sequences, active chromatin regions (Dnasel hypersensibility clusters or H3K27Ac Mark tracks) or transcription factor binding sites. The Gene Transcription Regulation Database (GTRD) [Yevshin et al., Nucleic Acids Res, 2017. 45(D1): p. D61-D67] was further used for identification of chromatin immunoprecipitation-DNA-sequencing (CHIP-seq) peaks, providing experimentally verified transcription factor binding sites. Together, this information enabled the inventors to highlight sequences in the above-identified regions with likely functional importance in GRM6 gene expression.

The inventors then selected three possible enhancer regions [407En(hGRM6), 444En(hGRM6) and 770En(hGRM6)] and two possible promoter regions [566P(hGRM6) and 454P(GRM6)] (FIG. 1 and Table 1) according to the following rationales: 407En(hGRM6) (−13873 to −13467 rel. TLSS GRM6) consists of the 300 bp conserved sequence between the murine and human genomes (horizontally striped in FIG. 1A). 770En(hGRM6) (−14236 to −13467 rel. TLSS GRM6) in addition to 407En(hGRM6) also contains the 3′ CHIP-seq peaks and Dnase hypersensibility cluster (−13990 to −13816 rel. TLSS GRM6). The 444En(hGRM6) (−14033 to −13590 rel. TLSS GRM6) is a 3′ and 5′ truncated version of 770En(hGRM6) including 3′ and 5′ only the ChiP-seq peaks.

When aligning the sequences −1000 to −1 (rel. TLSS) of GRM6 the inventors identified a 167 bp conserved region (−425 to −259 rel. TLSS GRM6) (FIG. 1B, horizontally striped in FIG. 1B). With this conserved sequence included, the inventors designed two promoters: 566P(hGRM6) (−691 to −126 rel. TLSS GRM6) containing in addition the 5′ transcription start site (TSS, −179 rel. TLSS GRM6) and the 5′ H3K27Ac Mark signal peak (−656 to −405 rel. TLSS GRM6) and the second 3′ ERG ChiP-Seq peak. ERG is known as an activator that interacts with FLI1 contained in 770En and 444En. The second selected promoter sequence 454P(hGRM6) (−453 to +1 rel. TLSS GRM6) extends further 5′ compared to 566P(hGRM6) including the TLSS and additional potentially regulatory sequences located between TLSS and TSS, such as the TCF7L1 and MYC ChiP-Seq peaks.

Five possible combinations of enhancer and promoter sequences (Table 1) preceding a reporter transgene were cloned between the ITR sequences of an adeno-associated viral (AAV) vector as detailed in the examples below using standard molecular methods:

TABLE 1
Selected enhancer/promoter combinations.

| Name | Length | Sequence N° |
|---|---|---|
| 407En_454P(hGRM6) | 867 bp | 11 |
| 444En_454P(hGRM6) | 917 bp | 15 |
| 407En_566P(hGRM6) | 978 bp | 12 |
| 770En_454P(hGRM6) | 1243 bp | 13 |
| 770En_566P(hGRM6) | 1354 bp | 14 |

### Example 3: Functional Promoter Evaluation in the Human Retina

Having the promoter designed on the human GRM6 gene in light of therapeutic use in human patients, all promoters were evaluated in post-mortem human retinal explants. For this, promoters were combined with a mCitrine transgene and packaged into self-complementary (sc) AAV capsids, in particular scAAV2(7m8) (Dalkara et al. Sci Transl Med 2013. 5: p. 189ra76).

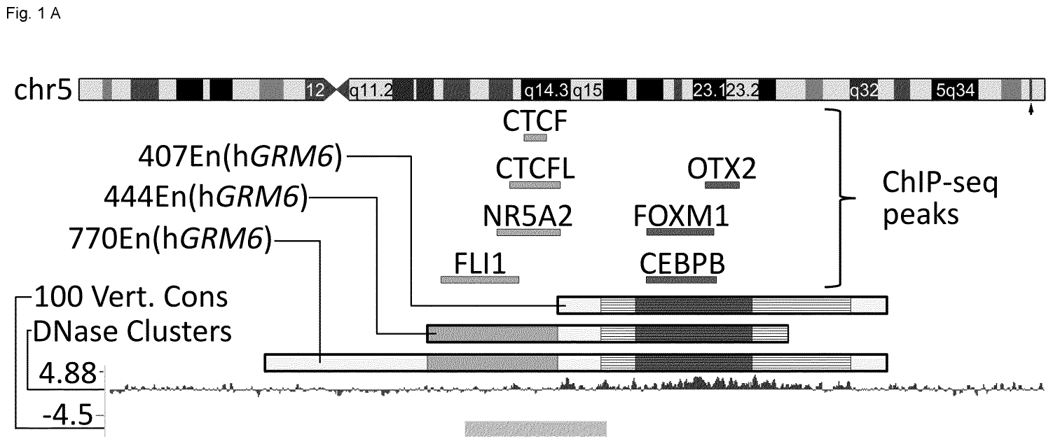

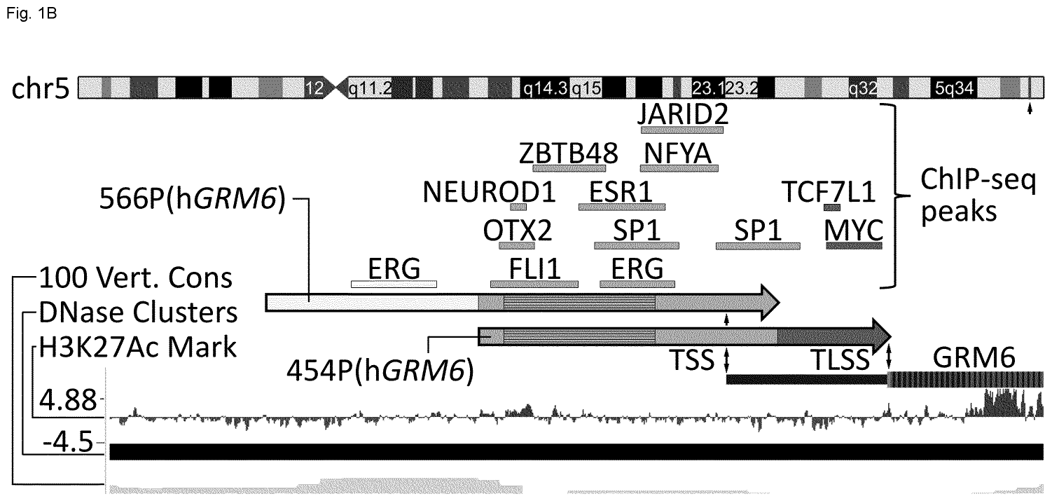

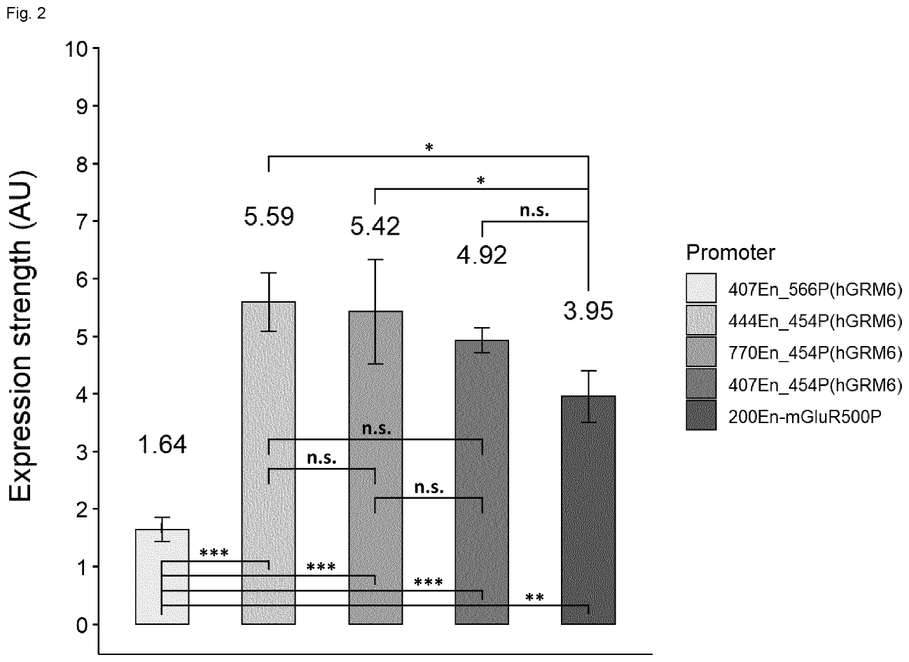

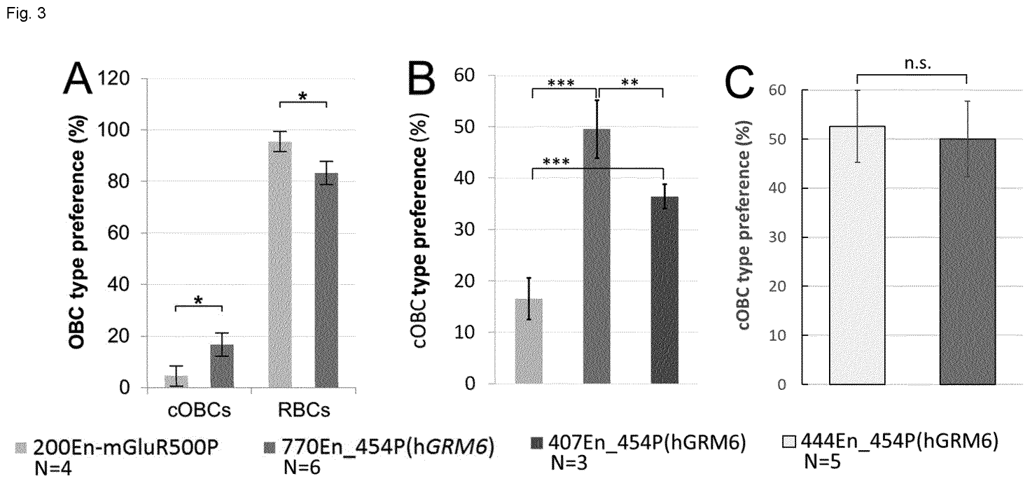

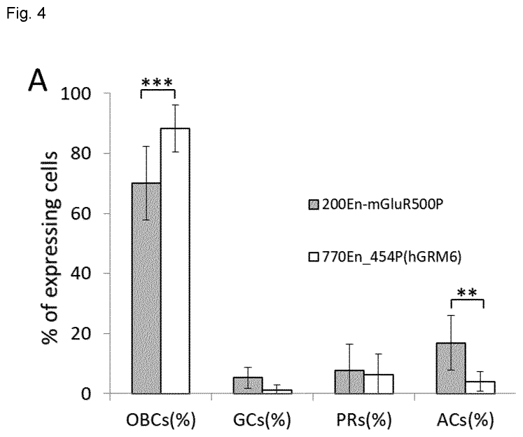

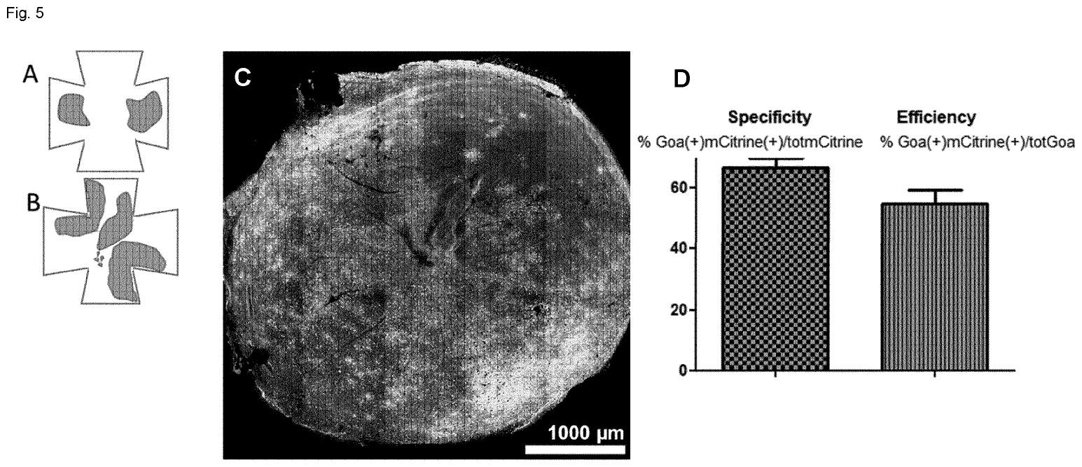

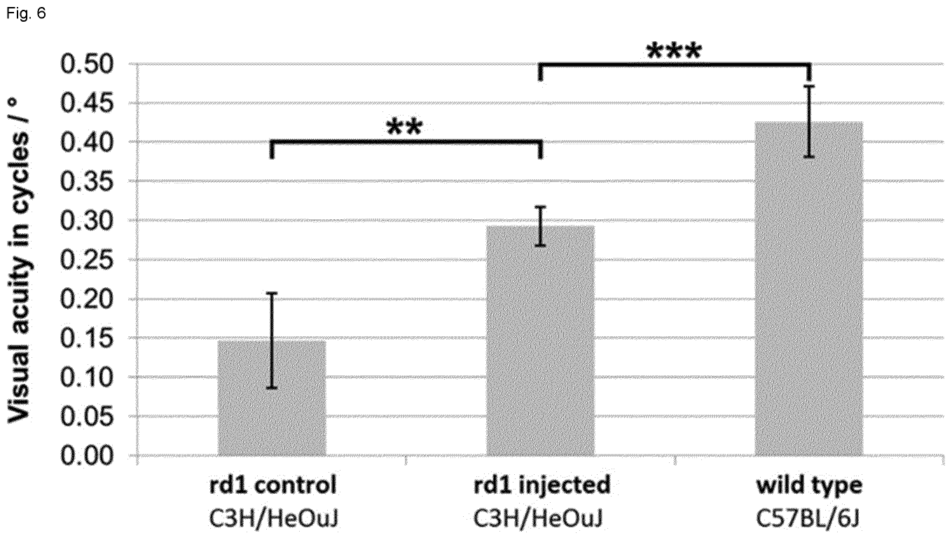

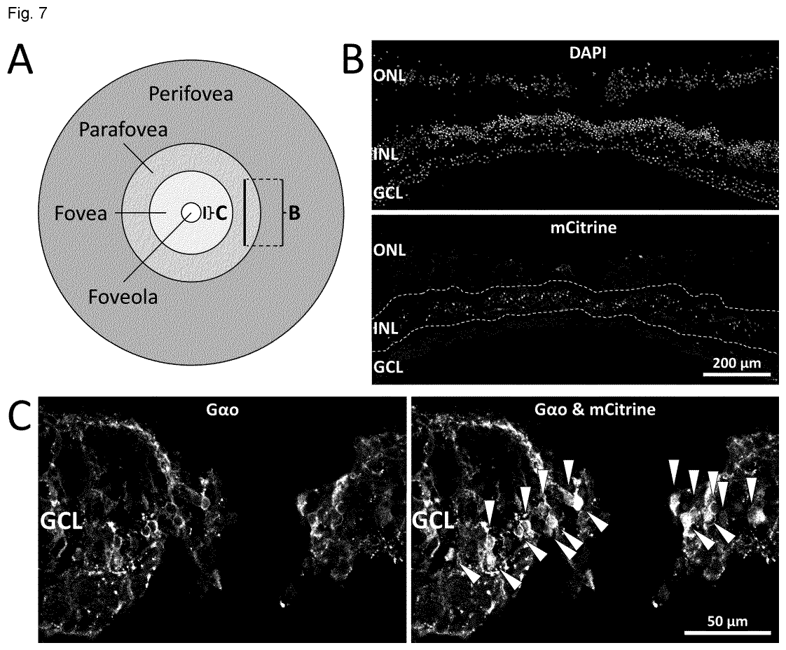

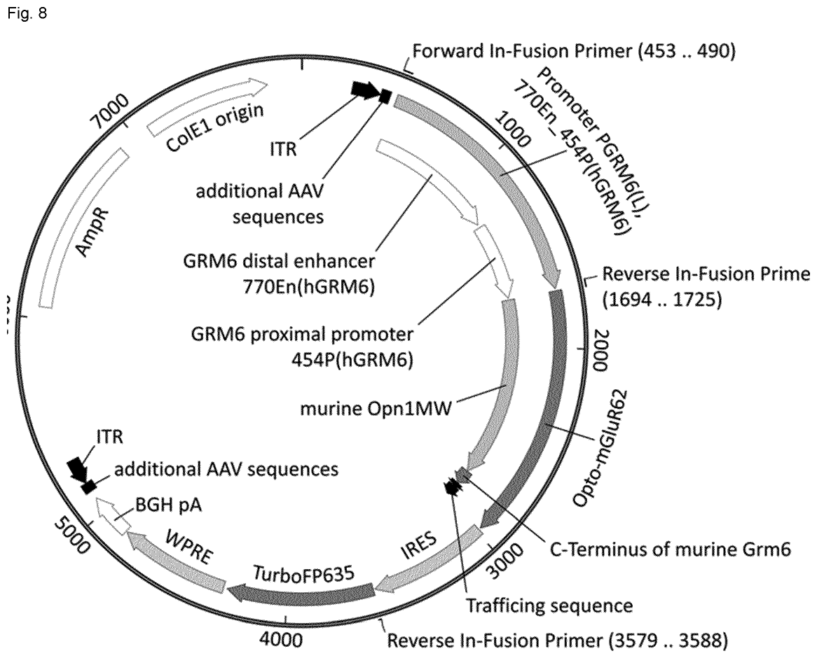

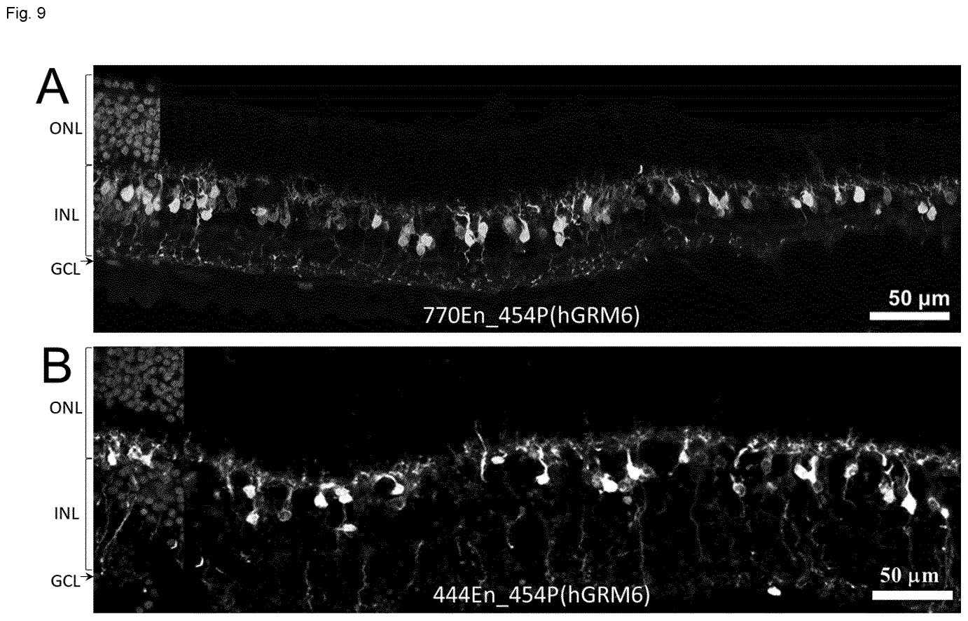

Approximately 5×10⁶ vg (vector genomes) were added to the RGC side of cultured post-mortem human retinal explants at day 1 as described in detail in [van Wyk, M., et al., Front Neurosci, 2017. 11(161): p. 161.]. Retinas were frozen at day 7 of culture when transgene expression from scAAVs was visible. The inventors stained cryo-sections for the reporter protein mCitrine and the OBC marker Goα to visualize localization of expression and to compare expression strengths. Up to 85% of OBCs were expressing mCitrine in well transduced areas (FIG. 9). To compare performance to state-of-the-art, Lu's 200En-mGluR500P promoter (Lu et al. Gene Ther, 2016. 23: p. 680-9.) was also packaged into scAAV2(7m8) and used in human retinal explant transduction. For each promoter, cultures from three human eyes were transduced with the promoter constructs and cryo-sections throughout the retina histologically stained and analysed. To control for variation between experiments, processing and immunohistochemistry were conducted for all samples in parallel. Fluorescence images were acquired with a ZEISS LSM 880 with Airyscan and ZEN 2.1 sofware. Confocal photomicrographs (z-stacks) were taken under a 20× objective with identical microscope settings. To determine average transduction efficacy, cytoplasmic Alexa488 fluorescence (secondary antibody for mCitrine) was determined from transduced OBC cell bodies [mCitrine(+) and Goα(+)]. In particular, arbitrary fluorescence values from a 4.15 μm diameter OBC somatic area were determined using the luminance function of Fiji image processing software. Image analyses, including fluorescence quantification and cell counting were performed with Fiji 21ulfils21 (version 2.0.0, https://fiji.sc/, Schindelin et al., Nat Methods, 2012. 9(7): p. 676-82). For normalization, the fluorescence value from each expressing OBC [mCitrine(+) and Goα(+)] was divided by the average background fluorescence value determined from measurements from non-expressing OBCs [mCitrine(−) and Goα(+)]. As evident from FIG. 2, the 566P basal promoter mediated weakest mCitrine expression in OBCs, whereas expression from 454P was always significantly stronger than that from Lu's 200En-mGluR500P, independent of the enhancer element employed. 454P was therefore selected for all subsequent experiments. 770En_454P(hGRM6) (F=6.03±1.53; mean±s.d.) and 407En_454P(hGRM6) (F=5.65±0.09; mean±s.d.) performed equally well in terms of efficacy and significantly better than Lu's mouse derived 200En-mGluR500P (F=4.49±0.22; mean±s.d.) and both were therefore analyzed in more detail.

### Example 4: Significantly Enhanced Cone ON-Bipolar Cell Preference in Human Retinal Explants

In a next step, sections were analysed for OBC cell-type specificity of expression. For this purpose, sections were stained with antibodies against the transgene mCitrine, the ubiquitous OBC markers Gαo and the rod bipolar cell specific antibody PKCα. [mCitrine(+), PKCα(+), Gαo(+)] cells were clearly identified as expressing rod ON-bipolar cells (RBCs), whereas [mCitrine(+), PKCα(−),Gαo(+)] cells were clearly identified as expressing cone ON-bipolar cells (cOBCs). Accordingly, [mCitrine(−), PKCα(+)] cells were identified as non-expressing RBCs and [mCitrine(−), PKCα(−), Gαo (+)] cells as non-expressing cOBCs. The results shown in FIG. 3 indicate clearly that both, 770En_454P(hGRM6) and 407En_454P(hGRM6) drive significantly higher transgene expression in cOBCs compared to 200En-mGluR500P. A measure for cOBC preference (FIG. 3B) was determined by normalizing the amount of expressing cOBCs and RBCs to the overall number of cOBCs and RBCs in the analysed retinal area. Such normalization revealed that 770En_454P(hGRM6) (cOBC preference of 49.5%) and 407En_454P(hGRM6) (cOBC preference of 36.4%) possessed a highly enhanced ability to drive expression in the cOBC cell type compared to 200En-mGluR500P (cOBC preference of 16.3%). Of particular note, 770En_454P(hGRM6) shows equal preference for RBCs and cOBCs (˜50% each, FIG. 3B).

Also the maculas of explanted human retinas were transduced with scAAV2(7m8)-770En_454P(hGRM6)-mCitrine. Immunolabeling with mCitrine, PKCα and Gαo clearly showed that the fovea contains exclusively cOBCs and that 770En_454P(hGRM6) drives mCitrine expression in virtually all cOBCs (FIG. 7). The ability to drive transgene expression efficiently in the cOBC of the human fovea is of major importance for a human therapy, since the fovea mediates high acuity vision and thus represents the primary target for a retinal gene therapy restoring vision—the fovea contains only cOBCs.

### Example 5: OBC Specificity of 770En_454P(hGRM6) in Comparison to 200En-mGluR500P

A high preference for OBCs is needed in order to avoid off-target effects such as corrupted retinal signaling. Human retinal sections were labelled with antibodies against mCitrine (transgene), Goα (general OBC marker) and the nuclear stain DAPI to differentiate cell layers. From this the identity of the expressing cell type could be derived: photoreceptors (PRs, mCitrine(+), located in the outer nuclear layer), OBCs [mCitrine(+),Goα(+) and located in the inner nuclear layer], amacrine cells [ACs, mCitrine(+),Goα(−) and located in the inner nuclear layer] and ganglion cells (GCs, mCitrine(+), located in the ganglion cell layer). FIG. 4A clearly shows that the novel promoter 770En_454P(hGRM6) has a significantly increased preference for OBCs (88.3±7.8%) compared to 200En-mGluR500P (70.1±12.2%). In addition, off-target expression of 200En-mGluR500P was generally higher, in particular in ACs (16.9±9.1%) as compared to the novel 770En_454P(hGRM6) promoter (4.1±3.2%). Latter is of particular importance for optogenetic vision restoration where off-target expression corrupts retinal signaling.

### Example 6: Promoter Evaluation in the Degenerating Mouse Retina

Important for a retinal therapy is the tissue's accessibility to treatment. This can be challenging in a degenerative process with anatomical, functional and transcriptional changes. The inventors had previously shown that Kim's murine 200En-SV40 promoter is no longer functional in the rapid degeneration rd1 mouse model (van Wyk et al. Front Neurosci 2017. 11: p. 161). Therefore, the performance of 770En_454P(hGRM6) [and for comparison its murine counterpart 200En-mGluR500P [Lu et al. Gene Ther 2016. 23 p: 680-689] were tested in the degenerative rd1 mouse model. The promoters were combined with the optogenetic MWOPN_-mGluR6-IRES2-TurboFP635 (SEQ ID NO: 16, plasmid map FIG. 8) transgene and packaged into ssAAV2(7m8) (Dalkara et al. Sci Transl Med 2013. 5: p. 189ra76). 3×10⁹ vg were intravitreally injected as described in (van Wyk et al. Front Neurosci 2017. 11: p. 161; van Wyk et al., PloS Biol 2015. 13: p. e1002143) into the eyes of late degenerated, 22 weeks old rd1 mice. 4 weeks post injections, mice were euthanized and retinas extracted for immunohistochemical analysis as previously described [van Wyk, M., et al., Front Neurosci, 2017. 11(161): p. 161 ;van Wyk et al., PloS Biol 2015. 13: p. e1002143]. The inventors labelled the sections against the transgene TurboFP635, the OBC-specific Goα marker and the nuclear stain DAPI. As opposed to 200En-SV40, 770En_454P(hGRM6) as well as Lu's Grm6-derived 200En-mGluR500P were functional in the OBCs of the rd1 retina (FIG. 5). However, 200En-mGluR500P led to expression only in restricted retinal areas, as exemplified in FIG. 5A, whereas 770En_454P(hGRM6) led to far more extensive and widespread transduction of the degenerated retina (FIG. 5B), probably due to its increased expression strength which overcomes expression threshold when some Grm6 downregulation has occurred. To analyze the specificity and efficacy of transgene (mCitrine) expression driven by 770En_454P(hGRM6) in a degenerated retina, the inventors injected an additional 9 rd1 mice at even later time points (29-33 weeks of age). After 4 weeks, animals were euthanized and eyes frozen and sectioned. Cryosections were again labeled with antibodies against mCitrine and Goα and analyzed under a confocal microscope. Even here, in these fully degenerated retinas, ˜67% of all cells expressing the transgene [mCitrine(+), Goα (−)] were OBCs [mCitrine(+), Goα (+)]. Equally, ˜55% of all OBCs [mCitrine(−), Goα (+)] expressed the transgene [mCitrine(+), Goα (+)] (FIGS. 5D). Widespread and specific expression of the therapeutic transgene in a degenerated retina is fundamental for an efficient gene therapy.

### Example 7: Optogenetic Gene Therapy and Vision Restoration in rd1 Mice

To see whether the favourable properties of 770En_454P(hGRM6) support functional optogenetic vision restoration targeted at the OBCs, the inventors performed a proof-of-principle experiment with the rd1 degeneration mouse model. The inventors injected 3×10⁹ vg of ssAAV(7m8)-770En_454P(hGRM6)-MWOPN_mGluR6-IRES2-TurboFP635-WPRE-BGHpA (plasmid map FIG. 8) bilaterally into three completely photoreceptor-less rd1 mice of 22 weeks of age. MWOPN_mGluR6 (SEQ ID NO 16) is a chimeric protein between murine cone middle-wavelength opsin (MWOPN) and murine mGluR6 that operates analogously to Opto-mGluR6 mediating OBC activity and with this visual restoration (van Wyk et al., PloS Biol 2015. 13: p. e1002143). The inventors measured visual acuity by detecting optomoter responses (OMRs) (after [Prusky et al., Invest Ophthalmol Vis Sci, 2004. 45(12): p. 4611-6.]) at different time points (days 41, 47, 55, 82 and 112) post injection in an automated virtual OptoDrum (Striatech®) containing small chamber (54×54×30 cm) with four screens (23.8″ full-HD IPS panels) surrounding a platform where an animal can be placed. The brightness of the screens was adjusted to 250 cd/m² and the bottom and the top of the chamber were covered with mirrors. A compact industrial camera (IR-sensitive 1/3″ CMOS sensor with global shutter and wide-angle lens, F1.6) tracked the head movements of mice from above while a rotating pattern of black and white non-sinusoidal vertical bars were displayed on the screen at different spatial frequencies. The software Optodrum analyzed the recorded head movements and controlled thickness, contrast and speed of the applied stimuli. The velocity of the moving bars was set to 12°/s and the contrast to 100%. As shown in FIG. 6, the median visual acuity for each mouse was determined from all measured visual acuities of the different trials. The median visual acuity of the treated mice was 0.29±0.03 cycles/° (n=3, ±s.d., P=0.0048), significantly better than that of the un-injected rd1 control mice with 0.15±0.06 cycles/° (n=7, P≤194) but still worse than that of the seeing C57BL/6 positive control, which had an average visual acuity of 0.43±0.05 cycles/° (n=10, s.d., P=0.0006). The optogenetic OBC targeted therapy, however, resulted in a marked improvement in the optomoter response. Together, the results prove that 770En_454P(hGRM6) fulfils all the requirements for an OBC-targeted human gene therapy, being a short (1243 bp), human gene based promoter, which in degeneration is still highly efficient and specific for OBCs, including cOBCs.

### Materials and Methods

### Bioactivity Assays

Bioactivity assays are described in the above Example sections. Culturing and AAV transduction of human retinal explants as well as intravitreal AAV injection into mouse eyes and subsequent immunohistochemical processing of frozen retinal sections is described in detail elsewhere [van Wyk, M., et al., Front Neurosci, 2017. 11(161): p. 161.].

### Determination of Cone ON-Bipolar Cell Specificity

For this purpose, retinal cryosections were triple-stained with antibodies against the transgene mCitrine (Invitrogen, A11122, 1:500), the ubiquitous OBC marker Gαo (EMD, MAB3073, 1:750) and the RBC specific antibody PKCα (Santa Cruz, sc8393, 1:750). [mCitrine(+), PKCα(+), Gαo(+)] cells were clearly identified as expressing RBCs, whereas [mCitrine(+), PKCα(−),Gαo(+)] cells were clearly identified as expressing cOBCs. OBC type preference depicted in FIG. 3A was determined by the ratios of expressing cOBCs to all OBCs [mCitrine(+), PKCα(−), Gαo(+)]/[Gαo(+)] and RBCs type preference by the ratio of expressing RBCs to all OBCs [mCitrine(+), PKCα(+), Gαo(+)]/[Gαo (+)]. To have a measure of cOBC preference of expression, in other words the chance that a cOBC expresses the inventors related the number of expressing cOBCs and RBCs, respectively, to the amount of cOBCs and RBCs in this particular retinal area of analysis. This normalization was possible, since [mCitrine(−), PKCα(+),Gαo(+)] cells can be clearly identified as non-expressing RBCs and [mCitrine(−), PKCα(−), Gαo(+)] as non-expressing cOBCs. The ratio [mCitrine(+), PKCα(+), Gαo13(+)]/[mCitrine(−), PKCα(+),Gαo(+)] therefore represents the percentage of transduced and expressing RBCs, whereas the ratio [mCitrine(+), PKCα(−),Gαo(+)]/[mCitrine(−), PKCα(−), Gαo(+)] represents the percentage of transduced and expressing cOBCs in the respective retinal area of analysis. Therefore, the resulting percentages shown in FIG. 3B indicate the chance that a cOBC gets transduced.

### Software Used for Molecular Engineering

The inventors used the Genome Browser of the Genomics Institute of the University of California Santa Cruz (UCSC Genome Browser, https://genome.ucsc.edu/) [Kent et al., Genome Res, 2002. 12(6): p. 996-1006; Kuhn et al., Brief Bioinform, 2013. 14(2): p. 144-61] to study genomic promoter sequences and genome annotations.

To identify transcription factors and transcription factor binding sites that are likely involved in the regulation of gene expression produced by the novel GRM6-based promoters the inventors employed ChIP-seq data from the Gene Transcription Regulation Database (GTRD, gtrd.biouml.org/) [Yevshin et al., Nucleic Acids Res, 2017. 45(D1): p. D61-D67]

Plasmid maps were generated in Vector NTI Advance (version 11.5.2)

### Antibodies

TABLE 3
Antibodies used for immunohistochemistry

| | | | | | | |
|---|---|---|---|---|---|---|
| Primary | 1_1 | anti-GFP | Rabbit | Invitrogen | A-11122 | 1:500 |
| antibodies | 1_2 | anti-GFP | Chicken | Abeam | ab13970 | 1:500 |
| | 1_3 | anti-tRFP | Rabbit | Evrogren | AB234 | 1:500 |
| | 1_4 | anti-PKCα | Mouse | Santa Cruz | sc-8393 | 1:750 |
| | | | | Biotechnology | | |
| | 1_5 | anti-Goa | Mouse | EMD Millipore | MAB3073 | 1:750 |
| | 1_6 | anti-Gγ13 | Rabbit | Santa Cruz | sc-368324 | 1:500 |
| | | | | Biotechnology | | |
| Secondary | 2_1 | anti-Rabbit | Goat | Invitrogen | A-11008 | 1:400 |
| antibodies | 2_2 | Alexa Fluor | Goat | Invitrogen | A-10521 | 1:400 |
| | | 488 | | | | |
| | | anti-Mouse | | | | |
| | | Cyanine 3 | | | | |
| | 2_3 | anti-Rabbit | Donkey | Invitrogen | A-21206 | 1:400 |
| | | Alexa Fluor | | | | |
| | | 488 | | | | |
| | 2_4 | anti-Chicken | Donkey | Jackson | 703-175-155 | 1:400 |
| | | Cyanine 5 | | Imm.Research | | |
| | | | | Laboratories | | |

### Confocal Imaging Hardware and Software

A ZEISS LSM 880 with Airyscan and ZEN 2.1 software was used to take confocal images with either a 20× or a 40× objective lens. Images were processed and evaluated in Fiji [Schindelin et al., Nat Methods, 2012. 9(7): p. 676-82.]. The cell counter plugin was used for cell counting and standard Fiji tools for image processing. The Stitch plugin [Preibisch et al., Bioinformatics, 2009. 25(11): p. 1463-5.] was used in cases where Fiji failed to automatically combine tile scan pictures.

### Statistics

If not stated otherwise, values were compared with a two-tailed Student's t-test and gave average values with ±the standard deviation across biological samples throughout this work. Significance levels are indicated by stars: * represents P≤0.05, ** represents P≤0.01 and *** represents P≤0.001.

### Other Methods

Remaining methods not described above and in examples 1 and 2 can be found in [van Wyk, M., et al., Front Neurosci, 2017. 11(161): p. 161.].

TABLE 4
Sequences
Sequences used in this study

| | SEQ ID | | |
|---|---|---|---|
| Name | NO. | Type | Origin |
| 407En(hGRM6) | 1 | nucleic acid | human |
| 770(6) | 2 | nucleic acid | human |
| 444En(hGRM6) | 3 | nucleic acid | human |
| 429En(mGrm6) | 4 | nucleic acid | murine |
| 792En(mGrm6) | 5 | nucleic acid | murine |
| 460En(mGrm6) | 6 | nucleic acid | murine |
| 454P(hGRM6) | 7 | nucleic acid | human |
| 566P(hGRM6) | 8 | nucleic acid | human |
| 454P(mGrm6) | 9 | nucleic acid | murine |
| 566P(m Grm6) | 10 | nucleic acid | murine |
| 407En_454P(hGRM6) | 11 | nucleic acid | human |
| 407En_566P(hGRM6) | 12 | nucleic acid | human |
| 770En_454P(hGRM6) | 13 | nucleic acid | human |
| 770En_566P(hGRM6) | 14 | nucleic acid | human |
| 444En_454P(hGRM6) | 15 | nucleic acid | human |
| MWOPN_mGluR6 | 16 | nucleic acid | murine |
| IRES2 | 17 | nucleic acid | Picornavirus |
| mCitrine | 18 | nucleic acid | Aequorea victoria |
| TurboFP635 | 19 | nucleic acid | Entacmaea quadricolor |
| WPRE | 20 | nucleic acid | Woodchuck hepatitis B |
| | | | virus |
| BGH pA | 21 | nucleic acid | bovine |
| sNRP-1 pA | 22 | nucleic acid | human |
| WT capsid AAV2 | 23 | amino acid | viral |
| KOZAK sequence a) | 24 | nucleic acid | eukaryotic |
| KOZAK sequence b) | 25 | nucleic acid | eukaryotic |

SEQ ID NO. 1: 407En(hGRM6)

| | | |
|---|---|---|
| 1 | ctctgatttt aaaggaagta gatacttcaa ataattcatc atggagtgca | 50 |
| 51 | atattttctg taggctttta gtagataact tcatcagttt aaagaagatc | 100 |
| 101 | cttagattat gaaacattta caattatgaa tgaatattag atgttatcaa | 150 |
| 151 | atgctttttc tgcatccatt tagataatca tgtttttcct ttaatctgtt | 200 |
| 201 | aatgcggtga attacattaa tagatttcct aagtcattaa tctgctaaag | 250 |
| 251 | tgcatttctg ggacaaacca gacttggtta tgacattgta tgtatttcag | 300 |
| 301 | tttgcaaata ttggactagg atttttgtat ctatattcct tagtttgacc | 350 |
| 351 | tgtaaatttt atttcttgta ctaagtatta gcctcacgaa aggcattgtc | 400 |
| 401 | aaatgtt | 407 |

SEQ ID NO. 2: 770En(hGRM6)

| | | |
|---|---|---|
| 1 | gggtctccaa cttgccaact gtagatcttg gaacctttca tccttcataa | 50 |
| 51 | ctgcataagc caattccttc taataaatct gtataatata tctgtctata | 100 |
| 101 | taataaatat gtacttacat aactctgtat gttacatcta tctattctat | 150 |
| 151 | ctatctatct atctatctat ctatctatct atctatcatc tatctatcta | 200 |
| 201 | tttatctatt ctctgtgtct ttggagaacc ctgacatagt aagcaatcat | 250 |
| 251 | atcacctgca aatgatgaaa gctgtgtatt ttccaaatca gtcgttttat | 300 |
| 301 | gtcttttttt cttgcactga ctagtgcccc ctagagggaa tgataattgg | 350 |
| 351 | aattattgtc ttgctctgat tttaaaggaa gtagatactt caaataattc | 400 |
| 401 | atcatggagt gcaatatttt ctgtaggctt ttagtagata acttcatcag | 450 |
| 451 | tttaaagaag atccttagat tatgaaacat ttacaattat gaatgaatat | 500 |
| 501 | tagatgttat caaatgcttt ttctgcatcc atttagataa tcatgttttt | 550 |
| 551 | cctttaatct gttaatgcgg tgaattacat taatagattt cctaagtcat | 600 |
| 601 | taatctgcta aagtgcattt ctgggacaaa ccagacttgg ttatgacatt | 650 |
| 651 | gtatgtattt cagtttgcaa atattggact aggatttttg tatctatatt | 700 |
| 701 | ccttagtttg acctgtaaat tttatttctt gtactaagta ttagcctcac | 750 |
| 751 | gaaaggcatt gtcaaatgtt | 770 |

SEQ ID NO. 3: 444En(hGRM6)

| | | |
|---|---|---|
| 1 | atctattctc tgtgtctttg gagaaccctg acatagtaag caatcatatc | 50 |
| 51 | acctgcaaat gatgaaagct gtgtattttc caaatcagtc gttttatgtc | 100 |
| 101 | tttttttctt gcactgacta gtgcccccta gagggaatga taattggaat | 150 |
| 151 | tattgtcttg ctctgatttt aaaggaagta gatacttcaa ataattcatc | 200 |
| 201 | atggagtgca atattttctg taggctttta gtagataact tcatcagttt | 250 |
| 251 | aaagaagatc cttagattat gaaacattta caattatgaa tgaatattag | 300 |
| 301 | atgttatcaa atgctttttc tgcatccatt tagataatca tgtttttcct | 350 |
| 351 | ttaatctgtt aatgcggtga attacattaa tagatttcct aagtcattaa | 400 |
| 401 | tctgctaaag tgcatttctg ggacaaacca gacttggtta tgac | 444 |

SEQ ID NO. 4: 429En(mGrm6), murine sequence corresponding to 407En(hGRM6)

| | | |
|---|---|---|
| 1 | aaaacatacc actttagttt aaggactata gtgattccac actaggtaag | 50 |
| 51 | gtgctttctg taggctttta gttaatagtt ttgtcaagct aaagaagatc | 100 |
| 101 | tccagatggc taaactttta aatcatgaat gaagtagata ttaccaaatt | 150 |
| 151 | gctttttcag catccattta gataatcatg ttttttgcct ttaatctgtt | 200 |
| 201 | aatgtagtga attacagaaa tacatttcct aaatcattac atcccccaaa | 250 |
| 251 | tcgttaatct gctaaagtac atctctggct caaacaagac tggttgtgac | 300 |
| 301 | aggtttgtct ctgtcagttt gtgactgttg ggctggctct tcctacccct | 350 |
| 351 | ctgcttcttg gtttggcctg aacattaatt ttattttatt tttttaattt | 400 |
| 401 | tacctacaat caatttcaca atgtgtgtt | 429 |

SEQ ID NO. 5: 792En(mGrm6), murine sequence corresponding to 770En(hGRM6)

| | | |
|---|---|---|
| 1 | ggtctcaaca agatacaaat tatgttctct aggtagcaat taacacaagg | 50 |
| 51 | aacgccttga ggtatgggag gggtgaggaa gctcacaaga tagaccctgg | 100 |
| 101 | tgcctggaag gaagacagcc aactaaaggt catatcacag tgtcccggga | 150 |
| 151 | accaacttga agggcttctg ctgtacaaat gtgggagaat ttcatcgtca | 200 |
| 201 | gaaggctctg caaaggtctg aaagtcaccg aactctgtaa gattctatcc | 250 |
| 251 | tgcttctatt cctgtcaaaa tataccagaa ggaatggaac taccccctcc | 300 |
| 301 | aaaaaataaa taaacaaaca aaccaccaaa ccacgcacag acaaagcatt | 350 |
| 351 | caatacacat gctaaaacat accactttag tttaaggact atagtgattc | 400 |
| 401 | cacactaggt aaggtgcttt ctgtaggctt ttagttaata gttttgtcaa | 450 |
| 451 | gctaaagaag atctccagat ggctaaactt ttaaatcatg aatgaagtag | 500 |
| 501 | atattaccaa attgcttttt cagcatccat ttagataatc atgttttttg | 550 |
| 551 | cctttaatct gttaatgtag tgaattacag aaatacattt cctaaatcat | 600 |
| 601 | tacatccccc aaatcgttaa tctgctaaag tacatctctg gctcaaacaa | 650 |
| 651 | gactggttgt gacaggtttg tctctgtcag tttgtgactg ttgggctggc | 700 |
| 701 | tcttcctacc cctctgcttc ttggtttggc ctgaacatta attttatttt | 750 |
| 751 | atttttttaa ttttacctac aatcaatttc acaatgtgtg tt | 792 |

SEQ ID NO. 6: 460En(mGrm6), murine sequence corresponding to 444En(hGRM6)

| | | |
|---|---|---|
| 1 | ggctctgcaa aggtctgaaa gtcaccgaac tctgtaagat tctatcctgc | 50 |
| 51 | ttctattcct gtcaaaatat accagaagga atggaactac cccctccaaa | 100 |
| 101 | aaataaataa acaaacaaac caccaaacca cgcacagaca aagcattcaa | 150 |
| 151 | tacacatgct aaaacatacc actttagttt aaggactata gtgattccac | 200 |
| 201 | actaggtaag gtgctttctg taggctttta gttaatagtt ttgtcaagct | 250 |
| 251 | aaagaagatc tccagatggc taaactttta aatcatgaat gaagtagata | 300 |
| 301 | ttaccaaatt gctttttcag catccattta gataatcatg ttttttgcct | 350 |
| 351 | ttaatctgtt aatgtagtga attacagaaa tacatttcct aaatcattac | 400 |
| 401 | atcccccaaa tcgttaatct gctaaagtac atctctggct caaacaagac | 450 |
| 451 | tggttgtgac | 460 |

SEQ ID NO. 7: 454P(hGRM6)

| | | |
|---|---|---|
| 1 | ggaggggtct ccaccctcgg agcggtctct catccctccc tagaatcctt | 50 |
| 51 | aaatcctctc tcgctcaggg cctcggccgc atctgtcaca gacttgtcct | 100 |
| 101 | gaaccgacag cggctggcgc aggtgactgg cttggggcgg gagcctgggt | 150 |
| 151 | gtgcgctggg gatggacccc gaggaagagg ggccaagctg tcgggaagcg | 200 |
| 201 | gcagggctgg aggggtggag gcagtggtcg ggcgggaccc cgggcgacag | 250 |
| 251 | ggttcggcgc ttgtaagagc gagacggagg cccgggcagg ccggctgagc | 300 |
| 301 | taactcccca gagccgaagt ggaaggcgcg ccccgagcgc cttctcccca | 350 |
| 351 | ggaccccggt gtccctcccc gcgccccgag cccgcgctct ccttcccccg | 400 |
| 401 | ccctcagagc gctccccgcc cctctgtctc cccgcagccc gctagacgag | 450 |
| 451 | ccga | 454 |

SEQ ID NO. 8: 566P(hGRM6)

| | | |
|---|---|---|
| 1 | ccaagaagag gacagaggca gaaagccagg gacagagact gagaaacaga | 50 |
| 51 | gacctagagg cagaagaaga ctgagataga gatggacaga gattgtgtca | 100 |
| 101 | gacacagccc cagagacagc cagacagtct gagtcagacg caaaccaaag | 150 |
| 151 | acaagaaaac aggaaaacag acccagagat tgggagaggg aggggaagga | 200 |
| 201 | gatgcgggga gagccagcac cgccaccccc cacactcagg aggggtctcc | 250 |
| 251 | accctcggag cggtctctca tccctcccta gaatccttaa atcctctctc | 300 |
| 301 | gctcagggcc tcggccgcat ctgtcacaga cttgtcctga accgacagcg | 350 |
| 351 | gctggcgcag gtgactggct tggggcggga gcctgggtgt gcgctgggga | 400 |
| 401 | tggaccccga ggaagagggg ccaagctgtc gggaagcggc agggctggag | 450 |
| 451 | gggtggaggc agtggtcggg cgggaccccg ggcgacaggg ttcggcgctt | 500 |
| 501 | gtaagagcga gacggaggcc cgggcaggcc ggctgagcta actccccaga | 550 |
| 551 | gccgaagtgg aaggcg | 566 |

SEQ ID NO. 9: 454P(mGrm6), murine sequence corresponding to 454P(hGRM6)

| | | |
|---|---|---|
| 1 | agagagaaga gagcccttcc tccactctca agctctggag ggggtctctg | 50 |
| 51 | ccctcaccct catccctccc cagaatcctt aaatcctcta gactgtagct | 100 |
| 101 | ctgattttac agctgtcaca gactcgtcct actagccaga ggttggctca | 150 |
| 151 | ggtaagcacc actggggagg tagcctaggg tgcgctgggg tgggtccaga | 200 |
| 201 | ggaagagctg cccagaactg tgggggaagg agcgggaccg accatcaaca | 250 |
| 251 | gggggacttt tcagggagaa tgagagcaat cctctggagg cctgggagag | 300 |
| 301 | gctgctgagt tgctggtgcg cgagtcacca acttttcctg cgctctcggt | 350 |
| 351 | gtccggccag aatcccgaag tggcagctga gcacggggtg gcagcttcgt | 400 |
| 401 | ccgccggctc tcaaggcgtc ccggtaactt cctttcccgc agtccaggag | 450 |
| 451 | caga | 454 |

SEQ ID NO. 10: 566P(mGrm6), murine sequence corresponding to 566P(hGRM6)

| | | |
|---|---|---|
| 1 | gaccgaccag gggagtccct ggacttcttt gttcctcttc tcggggtggc | 50 |
| 51 | gggactgatt gtgtaaatct cttatctcca actttcactc ttatctgtct | 100 |
| 101 | ctttaatcgg catattgagg atgagtggcc aagcttattg gtgttgctgg | 150 |
| 151 | gtcagacaat ttaaaggcag tctaggggag aagcagaccc agggagtcag | 200 |
| 201 | agaggcagag agagaagaga gcccttcctc cactctcaag ctctggaggg | 250 |
| 251 | ggtctctgcc ctcaccctca tccctcccca gaatccttaa atcctctaga | 300 |
| 301 | ctgtagctct gattttacag ctgtcacaga ctcgtcctac tagccagagg | 350 |
| 351 | ttggctcagg taagcaccac tggggaggta gcctagggtg cgctggggtg | 400 |
| 401 | ggtccagagg aagagctgcc cagaactgtg ggggaaggag cgggaccgac | 450 |
| 451 | catcaacagg gggacttttc agggagaatg agagcaatcc tctggaggcc | 500 |
| 501 | tgggagaggc tgctgagttg ctggtgcgcg agtcaccaac ttttcctgcg | 550 |
| 551 | ctctcggtgt ccggcc | 566 |

SEQ ID NO. 11: 407En_454P(hGRM6)

| | | |
|---|---|---|
| 1 | ctctgatttt aaaggaagta gatacttcaa ataattcatc atggagtgca | 50 |
| 51 | atattttctg taggctttta gtagataact tcatcagttt aaagaagatc | 150 |
| 101 | ettagattat gaaacattta caattatgaa tgaatattag atgttatcaa | 200 |
| 151 | atgctttttc tgcatccatt tagataatca tgtttttcct ttaatctgtt | 250 |
| 201 | aatgcggtga attacattaa tagatttcct aagtcattaa tctgctaaag | 300 |
| 251 | tgcatttctg ggacaaacca gacttggtta tgacattgta tgtatttcag | 350 |
| 301 | tttgcaaata ttggactagg atttttgtat ctatattcct tagtttgacc | 400 |
| 351 | tgtaaatttt atttcttgta ctaagtatta gcctcacgaa aggcattgtc | 450 |
| 401 | aaatgttgct agcggagggg tctccaccct cggagcggtc tctcatccct | 500 |
| 451 | ccctagaatc cttaaatcct ctctcgctca gggcctcggc cgcatctgtc | 550 |
| 501 | acagacttgt cctgaaccga cagcggctgg cgcaggtgac tggcttgggg | 600 |
| 551 | cgggagcctg ggtgtgcgct ggggatggac cccgaggaag aggggccaag | 650 |
| 601 | ctgtcgggaa gcggcagggc tggaggggtg gaggcagtgg tcgggcggga | 700 |
| 651 | ccccgggcga cagggttcgg cgcttgtaag agcgagacgg aggcccgggc | 750 |
| 701 | aggccggctg agctaactcc ccagagccga agtggaaggc gcgccccgag | 800 |
| 751 | cgccttctcc ccaggacccc ggtgtccctc cccgcgcccc gagcccgcgc | 850 |
| 801 | tctccttccc ccgccctcag agcgctcccc | 900 |
| 851 | cccgctagac gagccga | 867 |

SEQ ID NO. 12: 407En_566P(hGRM6)

| | | |
|---|---|---|
| 1 | ctctgatttt aaaggaagta gatacttcaa ataattcatc atggagtgca | 50 |
| 51 | atattttctg taggctttta gtagataact tcatcagttt aaagaagatc | 100 |
| 101 | ettagattat gaaacattta caattatgaa tgaatattag atgttatcaa | 150 |
| 151 | atgctttttc tgcatccatt tagataatca tgtttttcct ttaatctgtt | 200 |
| 201 | aatgcggtga attacattaa tagatttcct aagtcattaa tctgctaaag | 250 |
| 251 | tgcatttctg ggacaaacca gacttggtta tgacattgta tgtatttcag | 300 |
| 301 | tttgcaaata ttggactagg atttttgtat ctatattcct tagtttgacc | 350 |
| 351 | tgtaaatttt atttcttgta ctaagtatta gcctcacgaa aggcattgtc | 400 |
| 401 | aaatgttgct agccaagaag aggacagagg cagaaagcca gggacagaga | 450 |
| 451 | ctgagaaaca gagacctaga ggcagaagaa gactgagata gagatggaca | 500 |
| 501 | gagattgtgt cagacacagc cccagagaca gccagacagt ctgagtcaga | 550 |
| 551 | cgcaaaccaa agacaagaaa acaggaaaac agacccagag attgggagag | 600 |
| 601 | ggaggggaag gagatgcggg gagagccagc accgccaccc cccacactca | 650 |
| 651 | ggaggggtct ccaccctcgg agcggtctct catccctccc tagaatcctt | 700 |
| 701 | aaatcctctc tcgctcaggg cctcggccgc atctgtcaca gacttgtcct | 750 |
| 751 | gaaccgacag cggctggcgc aggtgactgg cttggggcgg gagcctgggt | 800 |
| 801 | gtgcgctggg gatggacccc gaggaagagg ggccaagctg tcgggaagcg | 850 |
| 851 | gcagggctgg aggggtggag gcagtggtcg ggcgggaccc cgggcgacag | 900 |
| 901 | ggttcggcgc ttgtaagagc gagacggagg cccgggcagg ccggctgagc | 950 |
| 951 | taactcccca gagccgaagt ggaaggcg | 867 |

SEQ ID NO. 13: 770En_454P(hGRM6)

| | | |
|---|---|---|
| 1 | gggtctccaa cttgccaact gtagatcttg gaacctttca tccttcataa | 50 |
| 51 | ctgcataagc caattccttc taataaatct gtataatata tctgtctata | 100 |
| 101 | taataaatat gtacttacat aactctgtat gttacatcta tctattctat | 150 |
| 151 | ctatctatct atctatctat ctatctatct atctatcatc tatctatcta | 200 |
| 201 | tttatctatt ctctgtgtct ttggagaacc ctgacatagt aagcaatcat | 250 |
| 251 | atcacctgca aatgatgaaa gctgtgtatt ttccaaatca gtcgttttat | 300 |
| 301 | gtcttttttt cttgcactga ctagtgcccc ctagagggaa tgataattgg | 350 |
| 351 | aattattgtc ttgctctgat tttaaaggaa gtagatactt caaataattc | 400 |
| 401 | atcatggagt gcaatatttt ctgtaggctt ttagtagata acttcatcag | 450 |
| 451 | tttaaagaag atccttagat tatgaaacat ttacaattat gaatgaatat | 500 |
| 501 | tagatgttat caaatgcttt ttctgcatcc ttacaattat tcatgttttt | 550 |
| 551 | cctttaatct gttaatgcgg tgaattacat atttagataa cctaagtcat | 600 |
| 601 | taatctgcta aagtgcattt ctgggacaaa taatagattt ttatgacatt | 650 |
| 651 | gtatgtattt cagtttgcaa atattggact ccagacttgg tatctatatt | 700 |
| 701 | ccttagtttg acctgtaaat tttatttctt aggatttttg ttagcctcac | 750 |
| 751 | gaaaggcatt gtcaaatgtt caattgatat gtactaagta gaggggtctc | 800 |
| 801 | caccctcgga gcggtctctc atccctccct aatgctagcg aatcctctct | 850 |
| 851 | cgctcagggc ctcggccgca tctgtcacag agaatcctta aaccgacagc | 900 |
| 901 | ggctggcgca ggtgactggc ttggggcggg acttgtcctg tgcgctgggg | 950 |
| 951 | atggaccccg aggaagaggg gccaagctgt agcctgggtg cagggctgga | 1000 |
| 1001 | ggggtggagg cagtggtcgg ccgggcaggc cggctgagct gttcggcgct | 1050 |
| 1051 | tgtaagagcg agacggaggc cccgagcgcc ttctccccag aactccccag | 1100 |
| 1101 | agccgaagtg gaaggcgcgc ccgcgctctc cttcccccgc gaccccggtg | 1150 |
| 1151 | tccctccccg cgccccgagc ccgcagcccg ctagacgagc cctcagagcg | 1200 |
| 1201 | ctccccgccc ctctgtctcc ccgcagcccg ctagacgagc cga | 1243 |

SEQ ID NO. 14: 770En_566P(hGRM6)

| | | |
|---|---|---|
| 1 | gggtctccaa cttgccaact gtagatcttg gaacctttca tccttcataa | 50 |
| 51 | ctgcataagc caattccttc taataaatct gtataatata tctgtctata | 100 |
| 101 | taataaatat gtacttacat aactctgtat gttacatcta tctattctat | 150 |
| 151 | ctatctatct atctatctat ctatctatct atctatcatc tatctatcta | 200 |
| 201 | tttatctatt ctctgtgtct ttggagaacc ctgacatagt aagcaatcat | 250 |
| 251 | atcacctgca aatgatgaaa gctgtgtatt ttccaaatca gtcgttttat | 300 |
| 301 | gtcttttttt cttgcactga ctagtgcccc ctagagggaa tgataattgg | 350 |
| 351 | aattattgtc ttgctctgat tttaaaggaa gtagatactt caaataattc | 400 |
| 401 | atcatggagt gcaatatttt ctgtaggctt ttagtagata acttcatcag | 450 |
| 451 | tttaaagaag atccttagat tatgaaacat ttacaattat gaatgaatat | 500 |
| 501 | tagatgttat caaatgcttt ttctgcatcc atttagataa tcatgttttt | 550 |
| 551 | cctttaatct gttaatgcgg tgaattacat taatagattt cctaagtcat | 600 |
| 601 | taatctgcta aagtgcattt ctgggacaaa ccagacttgg ttatgacatt | 650 |
| 651 | gtatgtattt cagtttgcaa atattggact aggatttttg tatctatatt | 700 |
| 701 | ccttagtttg acctgtaaat tttatttctt gtactaagta ttagcctcac | 750 |
| 751 | gaaaggcatt gtcaaatgtt caattgatat aatgctagcc aagaagagga | 800 |
| 801 | cagaggcaga aagccaggga cagagactga gaaacagaga cctagaggca | 850 |
| 851 | gaagaagact gagatagaga tggacagaga ttgtgtcaga cacagcccca | 900 |
| 901 | gagacagcca gacagtctga gtcagacgca aaccaaagac aagaaaacag | 950 |
| 951 | gaaaacagac ccagagattg ggagagggag gggaaggaga tgcggggaga | 1000 |
| 1001 | gccagcaccg ccacccccca cactcaggag gggtctccac cctcggagcg | 1050 |
| 1051 | gtctctcatc cctccctaga atccttaaat cctctctcgc tcagggcctc | 1100 |
| 1101 | ggccgcatct gtcacagact tgtcctgaac cgacagcggc tggcgcaggt | 1150 |
| 1151 | gactggcttg gggcgggagc ctgggtgtgc gctggggatg gaccccgagg | 1200 |
| 1201 | aagaggggcc aagctgtcgg gaagcggcag ggctggaggg gtggaggcag | 1250 |
| 1251 | tggtcgggcg ggaccccggg cgacagggtt cggcgcttgt aagagcgaga | 1300 |
| 1301 | cggaggcccg ggcaggccgg ctgagctaac tccccagagc cgaagtggaa | 1350 |
| 1351 | ggcg | 1354 |

SEQ ID NO. 15: 444En_454P(hGRM6)

| | | |
|---|---|---|
| 1 | atctattctc tgtgtctttg gagaaccctg acatagtaag caatcatatc | 50 |
| 51 | acctgcaaat gatgaaagct gtgtattttc caaatcagtc gttttatgtc | 100 |
| 101 | tttttttctt gcactgacta gtgcccccta gagggaatga taattggaat | 150 |
| 151 | tattgtcttg ctctgatttt aaaggaagta gatacttcaa ataattcatc | 200 |
| 201 | atggagtgca atattttctg taggctttta gtagataact tcatcagttt | 250 |
| 251 | aaagaagatc cttagattat gaaacattta caattatgaa tgaatattag | 300 |
| 301 | atgttatcaa atgctttttc tgcatccatt tagataatca tgtttttcct | 350 |
| 351 | ttaatctgtt aatgcggtga attacattaa tagatttcct aagtcattaa | 400 |
| 401 | tctgctaaag tgcatttctg ggacaaacca gacttggtta tgaccaattg | 450 |
| 451 | atataatgct agcggagggg tctccaccct cggagcggtc tctcatccct | 500 |
| 501 | ccctagaatc cttaaatcct ctctcgctca gggcctcggc cgcatctgtc | 550 |
| 551 | acagacttgt cctgaaccga cagcggctgg cgcaggtgac tggcttgggg | 600 |
| 601 | cgggagcctg ggtgtgcgct ggggatggac cccgaggaag aggggccaag | 650 |
| 651 | ctgtcgggaa gcggcagggc tggaggggtg gaggcagtgg tcgggcggga | 700 |
| 701 | ccccgggcga cagggttcgg cgcttgtaag agcgagacgg aggcccgggc | 750 |
| 751 | aggccggctg agctaactcc ccagagccga agtggaaggc gcgccccgag | 800 |
| 801 | cgccttctcc ccaggacccc ggtgtccctc cccgcgcccc gagcccgcgc | 850 |
| 851 | tctccttccc ccgccctcag agcgctcccc gcccctctgt ctccccgcag | 900 |
| 901 | cccgctagac gagccga | 917 |

SEQ ID NO. 16: MWOPN_mGluR6

| | | |
|---|---|---|
| 1 | atggcccaaa ggcttacagg tgaacagaca ctggaccact atgaggatag | 50 |
| 51 | cacccatgca agcatcttca cctataccaa cagcaacagc accaaaggtc | 100 |
| 101 | cctttgaagg ccccaattat cacattgctc ccaggtgggt gtaccacctc | 150 |
| 151 | accagcacct ggatgattct tgtggtcgtt gcatctgtct tcactaatgg | 200 |
| 201 | acttgtgctg gcagccacca tgagattcaa gaagctgcgc catccactga | 250 |
| 251 | actggattct ggtgaacttg gcagttgctg acctagcaga gaccattatt | 300 |
| 301 | gccagcacta tcagtgttgt gaaccaaatc tatggctact tcgttctggg | 350 |
| 351 | acaccctctg tgtgtcattg aaggctacat tgtctcattg tgtggaatca | 400 |
| 401 | caggcctctg gtccctggcc atcatttcct gggagagatg gctggtggtc | 450 |
| 451 | tgcaagccct ttggcaatgt gagatttgat gctaagctgg ccactgtggg | 500 |
| 501 | aatcgtcttc tcctgggtct gggctgctat atggacggcc ccaccaatct | 550 |
| 551 | ttggttggag caggtactgg ccttatggcc tgaagacatc ctgtggccca | 600 |
| 601 | gacgtgttca gcggtacctc gtaccccggg gttcagtctt atatgatggt | 650 |
| 651 | cctcatggtc acgtgctgca tcttcccact cagcatcatc gtgctctgct | 700 |
| 701 | acctccaagt gtggctggcc atccgagcag tggcaaagca acagaaagaa | 750 |
| 751 | tctgagtcca ctcagaaggc cgagaaggag gtgacacgca tggtggtggt | 800 |
| 801 | gatggtcttc gcatactgcc tctgctgggg accctatact ttctttgcat | 850 |
| 851 | gctttgctac tgcccaccct ggctatgcct tccaccctct tgtggcctcc | 900 |
| 901 | ctaccatcct actttgccaa aagtgccact atctacaacc ccattatcta | 950 |
| 951 | tgtctttatg aaccggcagt ttcgaaactg catcttacat ctctttggaa | 1000 |
| 1001 | agaaggttga tgatagctct gaactttcca gcacctccaa gacagaagtc | 1050 |
| 1051 | tcatctgtct cttcagtgtc acctgcagag cagaacgtgc agaagcggaa | 1100 |
| 1101 | gcgcagcctc aagaagacct ccacgatggc ggccccgccc aagagcgaga | 1150 |
| 1151 | actcagagga cgccaagaca gagaccagcc aagtggcgcc tgccaagagc | 1200 |
| 1201 | aggatcacca gcgagggcga gtacatcccc ctggaccaga tcgacatcaa | 1250 |
| 1251 | cgtgtaa | 1257 |

SEQ ID NO. 17: IRES2

| | | |
|---|---|---|
| 1 | gcccctctcc ctcccccccc cctaacgtta ctggccgaag ccgcttggaa | 50 |
| 51 | taaggccggt gtgcgtttgt ctatatgtta ttttccacca tattgccgtc | 100 |
| 101 | ttttggcaat gtgagggccc ggaaacctgg ccctgtcttc ttgacgagca | 150 |
| 151 | ttcctagggg tctttcccct ctcgccaaag gaatgcaagg tctgttgaat | 200 |
| 201 | gtcgtgaagg aagcagttcc tctggaagct tcttgaagac aaacaacgtc | 250 |
| 251 | tgtagcgacc ctttgcaggc agcggaaccc cccacctggc gacaggtgcc | 300 |
| 301 | tctgcggcca aaagccacgt gtataagata cacctgcaaa ggcggcacaa | 350 |
| 351 | ccccagtgcc acgttgtgag ttggatagtt gtggaaagag tcaaatggct | 400 |
| 401 | ctcctcaagc gtattcaaca aggggctgaa ggatgcccag aaggtacccc | 450 |
| 451 | attgtatggg atctgatctg gggcctcggt gcacatgctt tacatgtgtt | 500 |
| 501 | tagtcgaggt taaaaaaacg tctaggcccc ccgaaccacg gggacgtggt | 550 |
| 551 | tttcctttga aaaacacgat gataatatgg ccaca | 585 |

SEQ ID NO. 18: mCitrine

| | | |
|---|---|---|
| 1 | atggtgagca agggcgagga gctgttcacc ggggtggtgc ccatcctggt | 50 |
| 51 | cgagctggac ggcgacgtaa acggccacaa gttcagcgtg tccggcgagg | 100 |
| 101 | gcgagggcga tgccacctac ggcaagctga ccctgaagtt catctgcacc | 150 |
| 151 | accggcaagc tgcccgtgcc ctggcccacc ctcgtgacca ccttcggcta | 200 |
| 201 | cggcctgatg tgcttcgccc gctaccccga ccacatgaag cagcacgact | 250 |
| 251 | tcttcaagtc cgccatgccc gaaggctacg tccaggagcg caccatcttc | 300 |
| 301 | ttcaaggacg acggcaacta caagacccgc gccgaggtga agttcgaggg | 350 |
| 351 | cgacaccctg gtgaaccgca tcgagctgaa gggcatcgac ttcaaggagg | 400 |
| 401 | acggcaacat cctggggcac aagctggagt acaactacaa cagccacaac | 450 |
| 451 | gtctatatca tggccgacaa gcagaagaac ggcatcaagg tgaacttcaa | 500 |
| 501 | gatccgccac aacatcgagg acggcagcgt gcagctcgcc gaccactacc | 550 |
| 551 | agcagaacac ccccatcggc gacggccccg tgctgctgcc cgacaaccac | 600 |
| 601 | tacctgagct accagtccaa gctgagcaaa gaccccaacg agaagcgcga | 650 |
| 651 | tcacatggtc ctgctggagt tcgtgaccgc cgccgggatc actctcggca | 700 |
| 701 | tggacgagct gtacaagtcc ggataa | 726 |

SEQ ID NO. 19: TurboFP635

| | | |
|---|---|---|
| 1 | atggtgggtg aggatagcgt gctgatcacc gagaacatgc acatgaaact | 50 |
| 51 | gtacatggag ggcaccgtga acgaccacca cttcaagtgc acatccgagg | 150 |
| 101 | gcgaaggcaa gccctacgag ggcacccaga ccatgaagat caaggtggtc | 200 |
| 151 | gagggcggcc ctctcccctt cgccttcgac atcctggcta ccagcttcat | 250 |
| 201 | gtacggcagc aaaaccttta tcaaccacac ccagggcatc cccgacttct | 300 |
| 251 | ttaagcagtc cttccctgag ggcttcacat gggagaggat caccacatac | 350 |
| 301 | gaagacgggg gcgtgctgac cgctacccag gacaccagcc tccagaacgg | 400 |
| 351 | ctgcctcatc tacaacgtca agatcaacgg ggtgaacttc ccatccaacg | 450 |
| 401 | gccctgtgat gcagaagaaa acactcggct gggaggccag caccgagatg | 500 |
| 451 | ctgtaccccg ctgacagcgg cctgagaggc catagccaga tggccctgaa | 550 |
| 501 | gctcgtgggc gggggctacc tgcactgctc cctcaagacc acatacagat | 600 |
| 551 | ccaagaaacc cgctaagaac ctcaagatgc ccggcttcta cttcgtggac | 650 |
| 601 | aggagactgg aaagaatcaa ggaggccgac aaagagacct acgtcgagca | 700 |
| 651 | gcacgagatg gctgtggcca ggtactgcga cctgcctagc aaactggggc | 750 |
| 701 | acagctga | 708 |

SEQ ID NO. 20: WPRE

| | | |
|---|---|---|
| 1 | aatcaacctc tggattacaa aatttgtgaa agattgactg gtattcttaa | 50 |
| 51 | ctatgttgct ccttttacgc tatgtggata cgctgcttta atgcctttgt | 100 |
| 101 | atcatgctat tgcttcccgt atggctttca ttttctcctc cttgtataaa | 150 |
| 151 | tcctggttgc tgtctcttta tgaggagttg tggcccgttg tcaggcaacg | 200 |
| 201 | tggcgtggtg tgcactgtgt ttgctgacgc aacccccact ggttggggca | 250 |
| 251 | ttgccaccac ctgtcagctc ctttccggga ctttcgcttt ccccctccct | 300 |
| 301 | attgccacgg cggaactcat cgccgcctgc cttgcccgct gctggacagg | 350 |
| 351 | ggctcggctg ttgggcactg acaattccgt ggtgttgtcg gggaaatcat | 400 |
| 401 | cgtcctttcc ttggctgctc gcctgtgttg ccacctggat tctgcgcggg | 450 |
| 451 | acgtccttct gctacgtccc ttcggccctc aatccagcgg accttccttc | 500 |
| 501 | ccgcggcctg ctgccggctc tgcggcctct tccgcgtctt cg | 542 |

SEQ ID NO. 21: BGH pA

| | | |
|---|---|---|
| 1 | gcctcgactg tgccttctag ttgccagcca tctgttgttt gcccctcccc | 50 |
| 51 | cgtgccttcc ttgaccctgg aaggtgccac tcccactgtc ctttcctaat | 100 |
| 101 | aaaatgagga aattgcatcg cattgtctga gtaggtgtca ttctattctg | 150 |
| 151 | gggggtgggg tggggcagga cagcaagggg gaggattggg aagacaatag | 200 |
| 201 | caggcatgct gggga | 215 |

SEQ ID NO. 22: sNRP-1 pA

| | | |
|---|---|---|
| 1 | aaataaaata cgaaatg | 17 |

SEQ ID NO. 23: WT capsid AAV2

| | |
|---|---|
| MAADGYLPDW LEDTLSEGIR QWWKLKPGPP PPKPAERHKD DSRGLVLPGY KYLGPFNGLD | 60 |
| KGEPVNEADA AALEHDKAYD RQLDSGDNPY LKYNHADAEF QERLKEDTSF GGNLGRAVFQ | 120 |
| AKKRVLEPLG LVEEPVKTAP GKKRPVEHSP VEPDSSSGTG KAGQQPARKR LNFGQTGDAD | 180 |
| SVPDPQPLGQ PPAAPSGLGT NTMATGSGAP MADNNEGADG VGNSSGNWHC DSTWMGDRVI | 240 |
| TTSTRTWALP TYNNHLYKQI SSQSGASNDN HYFGYSTPWG YFDFNRFHCH FSPRDWQRLI | 300 |
| NNNWGFRPKR LNFKLFNIQV KEVTQNDGTT TIANNLTSTV QVFTDSEYQL PYVLGSAHQG | 360 |
| CLPPFPADVF MVPQYGYLTL NNGSQAVGRS SFYCLEYFPS QMLRTGNNFT FSYTFEDVPF | 420 |
| HSSYAHSQSL DRLMNPLIDQ YLYYLSRTNT PSGTTTQSRL QFSQAGASDI RDQSRNWLPG | 480 |
| PCYRQQRVSK TSADNNNSEF SWTGATKYHL NGRDSLVNPG PAMASHKDDE EKFFPQSGVL | 540 |
| IFGKQGSEKT NVDIEKVMIT DEEEIRTTNP VATEQYGSVS TNLQRGNRQA ATADVNTQGV | 600 |
| LPGMVWQDRD VYLQGPIWAK IPHTDGHFHP SPLMGGFGLK HPPPQILIKN TPVPANPSTT | 660 |
| FSAAKFASFI TQYSTGQVSV EIEWELQKEN SKRWNPEIQY TSNYNKSVNV DFTVDTNGVY | 720 |
| SEPRPIGTRY LTRNL | 735 |

SEQ ID NO 24
gccgccAccAUGG
SEQ ID NO 25
gccgccGccAUGG

1. An isolated nucleic acid molecule of 850 base pairs (bp) to 1500 bp length, comprising
a. an enhancer sequence element selected from SEQ ID NO 1 to 3, and
b. a promoter sequence element of SEQ ID NO 7.

2. An isolated nucleic acid molecule of 850 base pairs (bp) to 1500 bp length, comprising
a. an enhancer sequence element being at least (≥)70%, particularly ≥75%, ≥80%, more particularly ≥85%, more particularly ≥90%, more particularly ≥95%, even more particularly ≥98%, most particularly 100% identical to a sequence selected from SEQ ID NO 1 to 3; and
b. a promoter sequence element being ≥70%, particularly ≥75%, more particularly ≥80%, more particularly ≥85%, more particularly ≥90%, more particularly ≥95%, even more particularly ≥98%, most particularly 100% identical to a sequence of SEQ ID NO 7;
and said isolated nucleic acid molecule has ≥40%, particularly ≥50%, more particularly ≥60%, even more particularly ≥70%, more particularly ≥80%, even more particularly ≥90%, most particularly 100% of the cone ON bipolar cell-specificity from a sequence of SEQ ID NO 13 and a cone ON bipolar cell preference of ≥20%, particularly ≥25%, more particularly ≥30%, even more particularly ≥35%, more particularly ≥40%, most particularly ≥50%.

3. The isolated nucleic acid molecule according to claim 1 or 2, wherein the isolated molecule consists of one and only one of said enhancer sequence elements, one and only one of said promoter sequence elements and optionally, a spacer separating the enhancer sequence element from the promoter sequence element.

4. The isolated nucleic acid molecule according to any one of the preceding claims comprising or consisting of a sequence selected from SEQ ID NO 11, SEQ ID NO 13, and SEQ ID NO 15, or comprising or consisting of a sequence characterized by ≥98% identity to a sequence selected from SEQ ID NO 11, SEQ ID NO 13, and SEQ ID NO 15.

5. The isolated nucleic acid molecule according to any one of the preceding claims comprising or consisting of the sequence SEQ ID NO 11 or SEQ ID NO 13, or comprising or consisting of a sequence characterized by ≥98% identity to SEQ ID NO 11 or SEQ ID NO 13, particularly comprising or consisting of the sequence SEQ ID NO 13, or comprising or consisting of a sequence characterized by ≥98% identity to SEQ ID NO 13.

6. A nucleic acid expression vector comprising a nucleic acid molecule according to any one of the previous claims.

7. The nucleic acid expression vector according to claim 6, wherein the nucleic acid expression vector is an adeno-associated virus vector or a recombinant adeno-associated vector (rAAV), particularly wherein the nucleic acid expression vector is a recombinant AAV1, AAV2, AAV3, AAV4, AAV5, AAV6, AAV7, AAV8, AAV9, AAV10, AAV11, or AAV12 vector, more particularly wherein the nucleic acid expression vector is a recombinant AAV2 vector.

8. The nucleic acid expression vector according to any one of claims 6 to 7, additionally comprising
a. a sequence encoding a capsid protein, and
b. a transgene.

9. The nucleic acid expression vector according to claim 8, wherein the transgene comprises the sequence of SEQ ID NO 16.

10. An adeno-associated virion particle comprising the isolated nucleic acid molecule according to any one of claims 1 to 5 or the nucleic acid expression vector according to any one of claims 6 to 9.

11. An agent selected from the isolated nucleic acid molecule according to any one of claims 1 to 5 or the nucleic acid expression vector according to any one of claims 6 to 9, and the adeno-associated virion particle according to claim 10 for use as a medicament.

12. An agent selected from the isolated nucleic acid molecule according to any one of claims 1 to 5, the nucleic acid expression vector according to any one of claims 6 to 9, and the adeno-associated virion particle according to claim 10 for use in treatment of congenital stationary night blindness (CSBN1) or rod-cone and cone-rod dystrophies, particularly of retinitis pigmentosa and macular degeneration.

13. An agent selected from the isolated nucleic acid molecule according to any one of claims 1 to 5, the nucleic acid expression vector according to any one of claims 6 to 9, and the adeno-associated virion particle according to claim 10, wherein the agent is administered by
a. intravitreal administration, particularly by intravitreal injection, or by
b. subretinal injection.
